(19) United States
(12) Patent Application Publication
(10) Pub. No.: US 2024/0229092 A1
(43) Pub. Date: Jul. 11, 2024

(54) RESISTANT-ISOMALTO-OLIGOSACCHARIDE (IMO-R)

(71) Applicant: ADVENTUS LIFEFOODS INC., Edmonton (CA)

(72) Inventor: Mohammad Hassan QURESHI, Edmonton (CA)

(21) Appl. No.: 17/769,331

(22) PCT Filed: Dec. 16, 2020
(86) PCT No.: PCT/CA2020/051734

§ 371 Date: Apr. 14, 2022

Related U.S. Application Data
(60) Provisional application No. 62/950,354, filed on Dec. 19, 2019.

(51) Int. Cl. C12P 19/18; C07H 3/06; C12P 19/04; C12P 19/14

(52) U.S. Cl. CPC C12P 19/18; C07H 3/06; C12P 19/04; C12P 19/14

(57) ABSTRACT

A novel protocol is presented for the production of resistant-isomalto-oligosaccharides (“IMO-R”) that are resistant to human gut enzymes, in particular, dextranase, α-glucosidase and stomach and pancreatic α-amylases, thus exhibiting an increasing dietary Fiber effect.

### CROSS-REFERENCE TO RELATED APPLICATIONS

This application is a U.S. National Phase application of International Application No. PCT/CA2020/051734 filed 16 Dec. 2020, which claims priority of U.S. provisional patent application Ser. No. 62/950,354 filed Dec. 19, 2019, which is incorporated by reference into this application in its entirety.

### TECHNICAL FIELD

The present disclosure is related to the field of isomalto-oligosaccahrides (“IMOs”), in particular, resistant-IMOs that has an enhanced resistance towards human gut enzymes, thus exhibiting a prominent dietary fiber effect.

### BACKGROUND

The degree of resistance to enzymatic degradation of the current commercially available IMOs in human gastrointestinal (“GI”) system is significantly lower than expected or claimed by manufacturers. Evidence supporting the relatively lower functional potency of these commercial products prompted the United States Federal Drug Administration (“FDA”) in June 2018 to not include IMOs on the approved list of dietary fiber in June 2018, consequently resulting a huge negative impact on the IMO market. As a result, for instance, a Canada-based major IMO manufacturer submitted a petition to the FDA to have IMOs included on the list of dietary fiber in January 2019; the request was denied. Clearly, for IMOs to regain dietary fiber status, they must, at the very least, meet the expected degree of resistance to human gut enzymes as previously claimed by manufacturers of the currently available commercial products.

Generally, increasing the degree of branching in manufactured IMOs renders them increasingly resistant to human gut enzymes dextranase, α-glucosidase and stomach and pancreatic α-amylases and, therefore, increases their health functionalities as a functional health ingredient. IMOs of this category are generally referred to as resistant isomalto-oligosaccharides (IMO-Rs).

One school of thought is that the degree of branching and percentage of digestion-resistant linkages between the glucose units in the currently available commercial IMOs are not sufficiently high, which is why the products tend to have a relatively high susceptibility to GI enzymatic attack in the human gut. New strategies in the synthesis of IMOs are, therefore, needed to increase the degree of branching, percentage of digestion-resistant bonding within the oligosaccharide structure and functional potency of IMOs in order to meet expectations of the FDA for including it in the list of approved dietary fiber. There is no doubt that a “dietary fiber” designation will be a very strong market driver for the product which will re-invigorate the commercial IMO industry and create new business opportunities.

It is, therefore, desirable to provide IMOs that has increasing resistance to human gut enzymes, which can include, but are not limited to, dextranase, α-glucosidase and stomach & pancreatic α-amylases so as to improve the dietary-fiber functionality and, thus, called as a IMO-R.

### SUMMARY

A novel commercially production protocol for superior IMO-R is provided herein. In some embodiments, the protocol can comprise employing a series of carbohydrate specific enzymes with extensive degree of branching, linkage modifications, and cyclization within the oligo-structure for improved gut enzyme-resistance and dietary fiber functionality.

In some embodiments, the protocol can begin with an initial branching or saccharafication step by preparing a starting solution from a liquefied starch solution prepared from corn starch (same protocol is applicable to starch from other sources, i.e, Tapioca, Potato, Pea etc.). In some embodiments, first, the starting solution for the IMO-R production protocol can be prepared by adjusting the liquefied starch solution to a pH of about 6.3 to 6.7; then adjusting the temperature of the solution to about 63 to 67 degrees Celsius; then adding enzyme Branchzyme (“BE”) (Novozyme); then incubating with gentle stirring for a period of about 20 to 24 hours; then terminating the enzyme activity by bringing the temperature of the solution to that of boiling water for a period of about 30 minutes; and then cooling the resulting starting solution to room temperature.

In some embodiments, a first embodiment of the IMO-R protocol can comprise adjusting the pH of the starting solution to a range of about 4.5 to 5.5; then adjusting the temperature of the solution to a temperature in a range of about 50 to 55 degrees Celsius; then adding the enzyme Fungamyle 800L to the solution; then incubating the solution with gentle stirring for a period of about 30 minutes; then terminating the activity of the enzyme Fungamyle 800L by bringing the temperature of the solution to that of boiling water for a period of about 30 minutes; then adjusting the temperature of the solution to a temperature in a range of about 55 to 60 degrees Celsius; then adjusting the pH of the solution to a range of about 5.0 to 5.5; then adding the enzyme Transglucosidase (“TG”) to the solution; then incubating the solution with gentle stirring for a period of about 24 hours; and then terminating the activity of the enzyme TG by bringing the temperature of the solution to that of boiling water for a period of about 30 minutes. After the 24 hour incubation period, the final DE measurement of the solution is expected to be ≤50.

In some embodiments, a second embodiment of the IMO-R protocol can comprise adjusting the pH of the starting solution to a range of about 6.5 to 8.0; then adjusting the temperature of the solution to about 45 to 55 degrees Celsius; then adding the enzyme Glyco Transferase (“GTase”) (as manufactured by AMANO of Japan) to the solution; then incubating the solution with gentle stirring for a period of about 60 minutes; then adjusting the pH of the solution to a range of about 4.5 to 5.5; then adjusting the temperature of the solution to a temperature in a range of about 50 to 55 degrees Celsius; then adding the enzyme Fungamyle 800L to the solution; then incubating the solution with gentle stirring for a period of about 30 minutes; then terminating the activity of the enzymes by bringing the temperature of the solution to that of boiling water for a period of about 30 minutes; then cooling the temperature of the solution to a temperature in a range of about 55 to 60 degrees Celsius; then adjusting the pH of the solution to a range of about 5.0 to 5.5; then adding the enzyme TG to the solution; then incubating the solution with gentle stirring for a period of about 24 hours; and then terminating the activity of the enzyme TG by bringing the temperature of the solution to that of boiling water for a period of about 30 minutes. After the 24 hrs of incubation period, final DE measurement of the solution is expected to be ≤50.

In some embodiments, a third embodiment of the IMO-R protocol can comprise adjusting the pH of the starting solution to a range of about 6.5 to 8.0; then adjusting the temperature of the solution to about 45 to 55 degrees Celsius; then adding the enzyme GTase to the solution; then incubating the solution with gentle stirring for a period of about 60 minutes; then adjusting the pH of the solution to a range of about 5.0 to 6.0; then adjusting the temperature of the solution to a temperature in a range of about 80 to 90 degrees Celsius; then adding the enzyme Cycloglucanosyletransferase (“CGTase” also known as Toruzyme 3.0L as manufactured by Novozyme) to the solution; then incubating the solution with gentle stirring for a period of about 60 minutes; then adjusting the pH of the solution to a range of about 4.5 to 5.5; then adjusting the temperature of the solution to a range of about 50 to 55 degrees Celsius; then adding the enzyme Fungamyle 800L to the solution; then incubating the solution with gentle stirring for a period of about 30 minutes; then terminating the activity of the enzymes by bringing the temperature of the solution to that of boiling water for a period of about 30 minutes; then cooling the solution to a temperature in a range of about 55 to 60 degrees Celsius; then adjusting the pH of the solution to a range of about 5.0 to 5.5; then adding the enzyme TG to the solution; then incubating the solution with gentle stirring for a period of about 24 hours; and then terminating the activity of the enzyme TG by bringing the temperature of the solution to that of boiling water for a period of about 30 minutes. After the 24 hour incubation period, the final DE measurement of the solution is expected to be ≤50.

In some embodiments, the following commercially-available enzymes can be used in the protocol for the production of IMO-R product, it being understood by those skilled in the art that, for the purposes of this specification and of the claims that follow, other enzymes that are functionally and/or chemically equivalent as known to those skilled in the art can be substituted therefor or used in combination therewith and that the recitation of each of the following enzymes in said specification and said claims shall be interpreted as including all functional and/or chemical equivalents therefor either in substitution of, or in combination with, said following enzymes:
Enzyme A: Kleistase E5NC (AMANO-Japan)
Enzyme B: Fungamyle 800L (Novozyme)
Enzyme C: Branchzyme (BE)—29,400 U/g (Novozyme)
Enzyme D: Cycloglucanosyltransferase (CGTase)—3.41 U/g (Toruzyme 3.0L from Novozyme)
Enzyme E: Glyco Transferase Amano L (GTase)—3340 U/ml (Amano-Japan)
Enzyme F: Transglucosidase (TG) (Amano-Japan)

In some embodiments, the following materials can be used in the protocol:

Analytical standards (D-glucose, maltose monohydrate, maltotriose, panose, D-panose, isomaltotriose, α-cyclodextrin, β-cyclodextrin and γ-cyclodextrin) as sold by Sigma Aldrich of Oakville, Ontario, Canada; isomaltose, maltotetraose, isomaltotetraose, maltopentaose, maltohexaose and maltoheptaose as sold by Carbosynth, Compton, United Kingdom; methylene blue, potassium sodium tartrate tetrahydrate, sodium hydroxide, iodine and copper sulfate pentahydrate as sold by Fischer Scientific; Shodex Asahipak G220 HQ column as manufactured by Shodex of Japan and sold by Chromatographic Specialties of Brockville, Ontario, Canada; and Acetonitrile (“ACN”) and water (H₂O), both HPLC¹ grade, as sold by Fischer Scientific. Chemicals such as citric acid and potassium iodide can be used as well. ¹ High Performance Liquid Chromatography.

In some embodiments, the following materials can be used as well: liquified starch solutions processed from Tapioca starch; and Dietary Fiber Kits—AOAC 2011 & 2017, Integrated Dietary Fiber Kit (100 Assays), and Rapid Integrated Dietary Fiber Kit (100 Assays) as manufactured by Megazyme of Bray, Ireland.

In some embodiments, the following HPLC columns can be used: Carbohydrate Analysis column: Shodex Asahipak NH2P-50 4E—250×4.6 mm, 5 u Polymeric Amino column with an appropriate guard column; and Oligosaccharide profile and Dietary Fiber measurement: Phenomenex-Rezex-RSO-Oligosaccharide Ag+4%−2 00×10 mm HPLC Size Exclusion Column with an appropriate guard column.

Broadly stated, in some embodiments, a method can be provided for preparing a resistant-isomalto-oligosaccharide (“IMO-R”) with increasing resistance to human gut enzymes, the method comprising: adjusting the pH of a solution of liquified starch to about 4.5 to 5.5; then adding an effective amount of Fungamyle 800L to the solution; then incubating the solution at about 50 to 55 degrees Celsius for about 30 minutes, during which the solution is shaken or stirred; then terminating activity of the Fungamyle 800L by raising the temperature of the solution to that of boiling water for about 30 minutes; then cooling the solution to room temperature; then adjusting the pH of the solution to about 5.0 to 5.5; then adding an effective amount of Transglucosidase (“TG”) to the solution; then incubating the solution at about 55 to 60 degrees Celsius for about 24 hours, during which the solution is shaken or stirred; and then terminating activity of the TG by raising the temperature of the solution to that of boiling water for about 30 minutes.

Broadly stated, in some embodiments, the solution of liquified starch can be prepared by: mixing tapioca starch with water to form a starch slurry that is about 35% weight by volume; then adjusting the pH of the starch slurry to about 6.3 to 6.7 using a Na₂CO₃ solution dosed with an effective amount of Kleistase; then incubating the starch slurry at a temperature of about 87 degrees Celsius wherein an enzymatic reaction commences in the starch slurry; then checking the starch slurry for its Dextrose Equivalent (“DE”); then terminating the enzymatic reaction when the starch slurry is at a desired DE, wherein the enzymatic reaction is terminated by incubating the starch slurry at boiling water temperature for about 30 minutes and then cooling the starch slurry to room temperature; then adding an effective amount of Branchzyme (“BE”) to the starch slurry; then incubating the starch slurry by stirring it at about 63 to 67 degrees Celsius for about 24 hours; then terminating activity of the BE when the starch slurry achieves a DE of 20 by raising the temperature of the starch slurry to that of boiling water for about 30 minutes; and then cooling the starch slurry to room temperature thereby producing the solution of liquified starch.

Broadly stated, in some embodiments, the effective amount of the Kleistase can comprise 0.04% (v/w) thereof per gram of dry weight of starch.

Broadly stated, in some embodiments, the effective amount of the BE can comprise 600 Units (“U”) per gram of dry weight of starch.

Broadly stated, in some embodiments, the method can further comprise, prior to first adjusting the pH of the solution to about 4.5 to 5.5: adjusting the pH of the solution to about 6.5 to 8.0; then adding an effective amount of GlycoTransferase (“GTase”) to the solution; and then incubating the solution at about 45 to 55 degrees Celsius for about 60 minutes, during which the solution is shaken or stirred.

Broadly stated, in some embodiments, the method can further comprise, prior to first adjusting the pH of the solution to about 4.5 to 5.5: adjusting the pH of the solution to about 6.5 to 8.0, then adding an effective amount of Glyco Transferase (“GTase”) to the solution; then incubating the solution at about 45 to 55 degrees Celsius for about 60 minutes, during which the solution is shaken or stirred; and then, following step e) of claim 1, adjusting the pH of the solution to about 5.0 to 6.0, then adding an effective amount of Cycloglucanosyletransferase (“CGTase”) to the solution, and then incubating the solution at about 80 to 90 degrees Celsius for about 60 minutes, during which the solution is shaken or stirred.

Broadly stated, in some embodiments, the method can comprise, following the step of cooling the solution to room temperature: adjusting the pH of the solution to about 6.5 to 8.0, then adding an effective amount of Glyco Transferase (“GTase”) to the solution, and then incubating the solution at about 45 to 55 degrees Celsius for about 60 minutes, during which the solution is shaken or stirred.

Broadly stated, in some embodiments, the method can comprise: adjusting the pH of the solution to about 5.0 to 6.0; then adding an effective amount of Cycloglucanosyletransferase (“CGTase”) to the solution; and then incubating the solution at about 80 to 90 degrees Celsius for about 60 minutes, during which the solution is shaken or stirred.

Broadly stated, in some embodiments, the method can further comprise, prior to first adjusting the pH of the solution to about 4.5 to 5.5: adjusting the pH of the solution to about 6.5 to 8.0, then adding an effective amount of GlycoTransferase (“GTase”) to the solution, then incubating the solution at about 45 to 55 degrees Celsius for about 60 minutes, during which the solution is shaken or stirred, then adding an effective amount of Cycloglucanosyletransferase (“CGTase”) to the solution, and then incubating the solution at about 80 to 90 degrees Celsius for about 60 minutes, during which the solution is shaken or stirred.

Broadly stated, in some embodiments, a method can be provided for preparing a resistant-isomalto-oligosaccharide (“IMO-R”) with increasing resistance to human gut enzymes, the method comprising: adjusting the pH of a solution of liquified starch to about 6.5 to 8.0; then adding an effective amount of Glyco Transferase (“GTase”) to the solution; then incubating the solution at about 45 to 55 degrees Celsius for about 60 minutes, during which the solution is shaken or stirred; then adjusting the pH of the solution to about 5.0 to 6.0; then adding an effective amount of Cycloglucanosyletransferase (“CGTase”) to the solution; then incubating the solution at about 80 to 90 degrees Celsius for about 60 minutes, during which the solution is shaken or stirred; then adjusting the pH of the solution to about 5.0 to 5.5; then adding an effective amount of Transglucosidase (“TG”) to the solution; then incubating the solution at about 55 to 60 degrees Celsius for about 24 hours, during which the solution is shaken or stirred; and then terminating activity of the TG by raising the temperature of the solution to that of boiling water for about 30 minutes.

Broadly stated, in some embodiments, the effective amount of the Fungamyle 800L can comprise 0.8 millilitre thereof per kilogram of dry weight of starch.

Broadly stated, in some embodiments, the effective amount of the TG can comprise 1.4 millilitre thereof per kilogram of dry weight of starch.

Broadly stated, in some embodiments, the effective amount of the GTase can comprise 30 Units thereof per millilitre of the solution.

Broadly stated, in some embodiments, the effective amount of the CGTase can comprise 3.4 Units thereof per gram of dry weight of starch.

Broadly stated, in some embodiments, the human gut enzymes can comprise one or more of dextranase, α-glucosidase and α-amylase.

Broadly stated, in some embodiments, a human gut enzyme resistant-isomalto-oligosaccharide (“IMO-R”) can be provided, as produced by the methods described herein.

Broadly stated, in some embodiments, a method can be provided for preparing a resistant-isomalto-oligosaccharide (“IMO-R”) with increasing resistance to human gut enzymes, the method comprising the use of: Branchzyme (“BE”); GlycoTransferase (“GTase”); and Cycloglucanotransferase (“CGTase”) or Toruzyme 3.0L.

Broadly stated, in some embodiments, a human gut enzyme resistant isomalto-oligosaccharide (“IMO-R”) can be provided, as produced by a method comprising the use of: Branchzyme (“BE”); GlycoTransferase (“GTase”); and Cycloglucanotransferase (“CGTase”) or Toruzyme 3.0L.

### BRIEF DESCRIPTION OF THE DRAWINGS

FIG. 1 is a block diagram depicting seven enzymatic schemes for the synthesis of resistant isomalto-oligosaccharides (IMO-R).

FIG. 2 is an X-Y chart depicting a full chromatogram profile on HPLC with Phenomenex Rezex Column with liquified starch treated with Branchzyme (BE) for up to 24 hours.

FIG. 3 is an X-Y chart depicting a full chromatogram profile on HPLC with Phenomenex Rezex Column with liquified starch first treated with Branchzyme (BE), and then treated with Transglucosidase enzyme (TG).

FIG. 4 is an X-Y chart depicting a full chromatogram profile on HPLC with Shodex Column with liquified starch first treated with Branchzyme (BE), and finally treated with Transglucosidase enzyme (TG).

FIG. 5 is an X-Y chart depicting a full chromatogram profile on HPLC with Phenomenex Rezex Column with liquified starch first treated with Branchzyme (BE), and then treated with Fungamyle enzyme and finally treated with TG enzyme.

FIG. 6 is an X-Y chart depicting a full chromatogram profile on HPLC with Phenomenex Rezex Column with liquified starch first treated with Branchzyme (BE), and then treated with Fungamyle enzyme and finally with Transglucosidase (TG) enzyme.

FIG. 7 is an X-Y chart depicting a full Chromatogram profile on HPLC with Shodax Column with liquified starch first treated with Branchzyme (BE), and then treated with Fungamyle enzyme and finally with Transglucosidase (TG) enzyme.

FIG. 8 is an X-Y chart depicting a full chromatogram profile on HPLC with Phenomenex Rezex Column with liquified starch first treated with Branchzyme (BE), and then treated with GlycoTransferase (GTase), then treated with Fungamyle enzyme and finally with Transglucosidase (TG) enzyme.

FIG. 9 is an X-Y chart depicting a full chromatogram profile on HPLC with Showa Asahi pack—Shodax Column with liquified starch first treated with Branchzyme (BE), and then treated with Glyco Transferase (GTase), then treated with Fungamyle enzyme and finally with Transglucosidase (TG) enzyme.

FIG. 10 is an X-Y chart depicting a full chromatogram profile on HPLC with Phenomenex Rezex Column with liquified starch first treated with Branchzyme (BE), and then treated with GlycoTransferase (GTase), then treated with Fungamyle enzyme, then treated cyclo-glucanotransferase (CGTase), and finally with Transglucosidase (TG) enzyme.

FIG. 11 is an X-Y chart depicting a full chromatogram profile on HPLC with Phenomenex Rezex Column with liquified starch first treated with Branchzyme (BE), and then treated with Glyco Transferase (GTase), then treated with Fungamyle enzyme, then treated cyclo-glucanotransferase (CGTase), and finally with Transglucosidase (TG) enzyme.

FIG. 12 is an X-Y chart depicting a full chromatogram profile on HPLC with Showa Asahi pack—Shodex Column with liquified starch first treated with Branchzyme (BE), and then treated with GlycoTransferase (GTase), then treated with Fungamyle enzyme, then treated cyclo-glucanotransferase (CGTase), and finally with Transglucosidase (TG) enzyme.

FIG. 13 is an X-Y chart depicting a full chromatogram profile on HPLC with Phenomenex Rezex Column with liquified starch first treated with Branchzyme (BE), and then treated with Fungamyle enzyme, then treated with GlycoTransferase (GTase), and finally with Transglucosidase (TG) enzyme.

FIG. 14 is an X-Y chart depicting a full chromatogram profile on HPLC with Showa Asahi pack—Shodax Column with liquified starch first treated with Branchzyme (BE), and then treated with Fungamyle enzyme, then treated with GlycoTransferase (GTase), and finally with Transglucosidase (TG) enzyme.

FIG. 15 is an X-Y chart depicting a full chromatogram profile on HPLC with Phenomenex Rezex Column with liquified starch first treated with Branchzyme (BE), and then treated with GlycoTransferase (GTase), then treated with CGTase enzyme, and finally with Transglucosidase (TG) enzyme.

FIG. 16 is an X-Y chart depicting a full chromatogram profile on HPLC with Showa Asahi pack—Shodax Column with liquified starch first treated with Branchzyme (BE), and then treated with GlycoTransferase (GTase), then treated with CGTase enzyme, and finally with Transglucosidase (TG) enzyme.

FIG. 17 is an X-Y chart depicting a full chromatogram profile on HPLC with Phenomenex Rezex Column with liquified starch first treated with Branchzyme (BE), and then treated with with GlycoTransferase (GTase), then treated with CGTase enzyme, then treated with Fungamyle, and finally with Transglucosidase (TG) enzyme.

FIG. 18 is an X-Y chart depicting a full chromatogram profile on HPLC with Shodax Column with liquified starch first treated with Branchzyme (BE), and then treated with GlycoTransferase (GTase), then treated with CGTase enzyme, then treated with Fungamyle, and finally with Transglucosidase (TG) enzyme.

FIG. 19 is an X-Y chart depicting a full chromatogram profile on HPLC with Phenomenex Rezex Column with liquified starch first treated with Branchzyme (BE), and then treated with Fungamyle, then treated with Glyco Transferase (GTase), then treated with CGTase enzyme, and finally with Transglucosidase (TG) enzyme.

FIG. 20 is an X-Y chart depicting a full chromatogram profile on HPLC Shodex Column with liquified starch first treated with Branchzyme (BE), and then treated with Fungamyle, then treated with GlycoTransferase (GTase), then treated with CGTase enzyme, and finally with Transglucosidase (TG) enzyme.

FIG. 21 is an X-Y chart depicting a full chromatogram profile on HPLC-RI with Showa Asahi pack—Shodex Column.

FIG. 22 is an X-Y chart depicting a full chromatogram profile on HPLC with Phenomenex Zerbex Column.

FIG. 23 is an X-Y chart depicting a full chromatogram profile on HPLC with Phenomenex Zerbex Column after treating the IMO-R with pancreatic alpha-amylase (PAA), and amyloglucosidase (AMG) for 16 hours, as per protocol of AOAC-2011 standard method (in-house TDF testing).

FIG. 24 is an X-Y chart depicting a comparison of before and after glucose filtration from Final IMO-R sample.

FIG. 25 is an X-Y chart depicting a comparison of oligo profile before and after filtration.

FIG. 26 is an X-Y chart depicting HPLC-RI (with Rezex-Column) TDF profiles of sample after post-amylase hydrolysis using AOAC-2011 method.

FIG. 27 is and HPLC-RI (with Rezex-Column) TDF profiles of sample after post-amylase hydrolysis using AOAC-2017 method.

### DETAILED DESCRIPTION OF EMBODIMENTS

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment can also be included in other embodiments but is not necessarily included. Thus, the present technology can include a variety of combinations and/or integrations of the embodiments described herein.

A novel commercial manufacturing protocol for superior Resistant-IMO (IMO-R) is provided herein.

In some embodiments, the enzymes that can be used in the enzymatic synthesis of IMO-R are shown in Table 1.

| S. No | Enzyme Name | Supplier | Properties |
|---|---|---|---|
| 1. | Kleistase (E5NC) | L-Amano | This enzyme is a bacterial endo alpha-amylase |
| | | | derived from Bacillus amyloliquefaciens. It |
| | | | catalyzes the hydrolysis of α(1,4)-glycosidic |
| | | | linkages of gelatinous starch randomly to |
| | | | produce soluble maltodextrins, reducing the |
| | | | high viscosity of starch slurry and decolorizing |
| | | | the blue color of starch-iodine complex. |
| 2. | Branchzyme (BE) | Novozymes | BE catalyzes the synthesis of α(1,6)-glycosidic |
| | | | linkages, thereby increasing their ratio with |
| | | | respect to α(1,4)-glycosidic linkages. Its |
| | | | mechanism of action leading to rearrangement |
| | | | of side-chains involves the cleaving of α(1,4) |
| | | | linkages and transferring the moieties to |
| | | | generate new α(1,6) linkages which increases |
| | | | the degree of branching in the α-limit dextrin |
| | | | mixture. This is because α(1,6)-glycosidic |
| | | | linkages are less digestible than α(1,4) linkages |
| | | | in human digestive tracts. |
| 3. | GlycoTransferase | L-Amano | In glycogen, every 10 to 14 glucose units, a side |
| | (GTase) | (Japan) | branch with an additional chain of glucose units |
| | | | occurs. The side chain attaches at carbon atom |
| | | | 6 of a glucose unit, an α(1,6)-glycosidic bond. |
| | | | This connection is catalyzed by a branching |
| | | | enzyme - GTase, generally given the name α- |
| | | | glucan branching enzyme. A branching enzyme |
| | | | attaches a string of seven glucose units (with |
| | | | some minor variation to this number) to the |
| | | | carbon at the C-6 position on the glucose unit, |
| | | | forming the α(1,6)-glycosidic bond. The |
| | | | specific nature of this enzyme means that this |
| | | | chain of 7 carbons is usually attached to a |
| | | | glucose molecule that is in position three from |
| | | | the non-reducing end of another chain. Because |
| | | | the enzyme works with such specificity |
| | | | regarding the number of glucose units |
| | | | transferred and the position to which they are |
| | | | transferred, the enzyme creates the very |
| | | | characteristic, highly branched glycogen |
| | | | molecule which is resistant in digestion in small |
| | | | intestine. |
| 4. | Cyclo- | Novozymes | Catalyzes the formation of α-and β- |
| | glucanotransferase | | cyclodextrins of various sizes of starch and |
| | (CGTase - | | similar substrates. |
| | Toruzyme 3.0 L) | | |
| 5. | Fungamy1 800 L | Novozymes | The enzyme hydrolyzes α(1,4)-glycosidic |
| | | | linkages in amylose and amylopectin; a |
| | | | prolonged reaction results in the formation of |
| | | | large amounts of maltose. In the starch industry, |
| | | | Fungamy1 800 L is used for production of high |
| | | | maltose syrups, 45-60% maltose (2-7% |
| | | | glucose). |
| 6. | Transglucosidase | L-Amano | TG acts in a two-step mechanism involving an |
| | (TG) | (Japan) | α-(1,4)-glycoside hydrolysis on glycoside |
| | | | donor and thereafter, transfer of the cleaved |
| | | | glucosyl group to position 6 of the glycoside |
| | | | acceptor to form an α(1,6)-glycosyl linkage. |

TABLE 1

### EXPERIMENTAL PROCEDURES

In some embodiments, the steps of the protocol to produce IMO-R can be summarized as follows. In some embodiments, the protocol can begin with a starting liquified starch solution, which can be prepared as follows.

### Step 1—Preparation of Liquified Starch

A 35% (w/v) of Tapioca starch slurry (175 g starch/500 ml of water) can be prepared by adjusting its pH to 6.0 to 6.5 using 3% Na2CO3 solution dosed with 70 μL of Kleistase enzyme (required concentration—0.04% (v/w) of the enzyme per gram of dry weight of starch). For example, for 175 g of starch, then 0.04% (v/w) of Kleistase would represent 175×0.04/100=0.07 millilitre or 70 μL of Kleistase added to the 175 g starch slurry. This slurry can be incubated at 87° C. at 150 rpm with an impeller programmable mixer. The enzymatic reaction can be performed for 25 min and checked for Starch content by Starch-Iodine method (see Appendix-6) for every 5 min till the blue color disappears and desired Dextose Equivalent (“DE”) reaches 12±2 using the Lane Enyon method. Then the reaction can be terminated by incubating the reaction mass at 100° C. for 30 min and cooled to room temperature.

In other embodiments, a ready-made liquified starch solution can be obtained from commercial sources, as well known to those skilled in the art. In that case, the liquified starch solution comprises of the following properties:
a) Starch source=Tapioca
b) Solid content=33.3%
c) pH=1.7
d) DE=11.4

### Step 2—Preparation of Post-Brachzyme (“BE”) Treated Solution

In some embodiments, the liquified starch solution can then be treated with Branchzyme (“BE”) (as sourced from Novozyme). Initially, the liquefied starch pH was 1.7 and can be adjusted to about 6.3 to 6.7 using 3% NaOH with constant stirring. Then, 460 mL of liquefied starch can be dosed with 2.73 g of Branchzyme, which is equivalent to loading of 600 U/g of starch (Actual enzyme concentration 29,500 U/g solution). The reaction can then be incubated at about 63 to 67° C. in a shaking water bath at 60 rpm. Thus, the liquified starch solution changed from milky slurry to a clear brown solution with very less solids settling at the bottom. After 24 hrs, a sample was taken and analyzed for DE till it reaches to DE=20. The reaction can then be terminated by incubating the flask at boiling water temperature for 30 min for enzyme denaturation. The resulting post-BE treated solution can be divided into several equal portions for subsequent treating with set of additional branching enzymes, which are explained as follows.

### Step 3—Designing of Enzymatic Schemes to Produce IMO-R

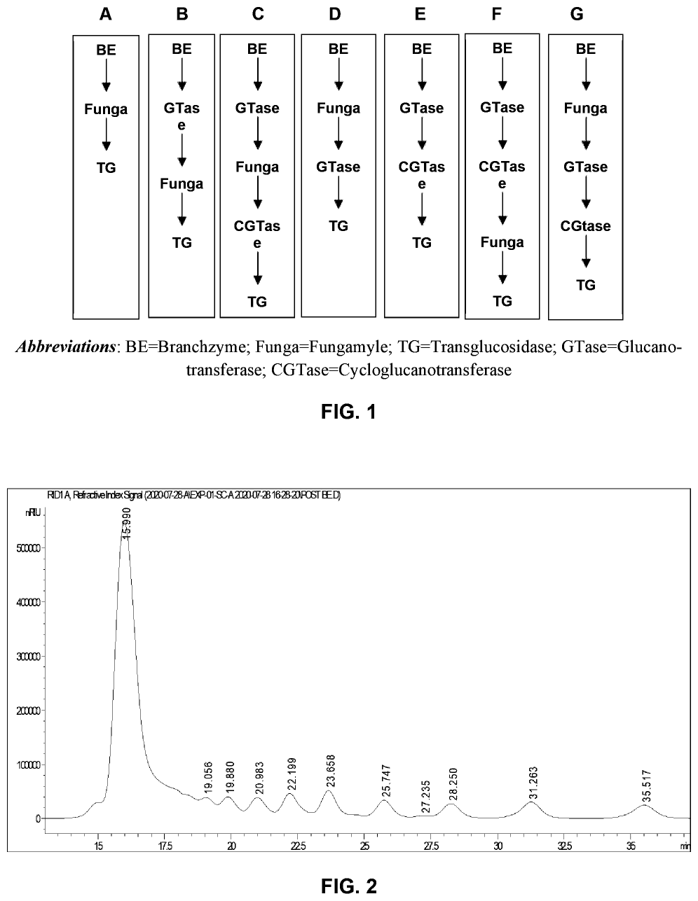

For the post-BE processing steps to produce IMO-R, in some embodiments, there can be seven enzymatic schemes designed and employed, which are represented herein as A, B, C, D, E, F & G (as shown in FIG. 1 and Table 2). Each scheme was carefully designed with a set of enzymes and specific arrangement. The purpose was to maximize branching level, and dietary fiber value. Samples are collected and tested at specific enzymatic step within each scheme and tested for general carbohydrate profile, IMO-specific oligo profile, and total dietary fiber content by using HPLC-RI and AOAC 2009/2017 methodologies, respectively. Further, the glucose contaminant in the final product was removal by using a nano-filtration device equipped with a specific molecular-weight-cut-off (“MWCO”) filtration membranes (Refer to Appendix 5).

In addition to the 7 schemes mentioned above, an extension of Scheme E and F were conducted with increasing level of enzymes GTase and CGTase. The first step, and second step increments of enzymes called as Scheme E (Modi-1), Scheme-E (Modi-2), and Scheme-F (Modi-1), Scheme-F (Modi-2). The purpose of doing so is to see the impact of increased level of branching enzymes upon carbohydrate profiles and dietary fiber content (see Table 3).

### Step 4—Detailed Protocol of Enzymatic Schemes

### Scheme-A (2 Stages)

In some embodiments, the protocol of Scheme-A can comprise the following stages:

### Stage-1:

To 50 mL of the post BE solution, adjust pH to 4.5 to 5.5 by 3% Citric Acid solution.
Add Fungamyl enzyme (0.8 mL/Kg dry weight of starch) (Actually, 50 mL of BE step solution is dosed with 13.3 μL of Fungamyl Enzyme).
Put the reaction solution in shaking water bath set at 53° C. Maintain the reaction at 50 to 55° C. for 20 min in shaking water bath.
At exactly 20 min, terminate the reaction by placing the solution flask in boiling water bath for 30 min.
Cool the solution to room temperature and sample obtained for HPLC-RI and DE analysis.
Proceed to Stage-2.

### Stage-2:

To Solution from Stage-1; adjust the pH to 5.0 to 5.5 using 3% citric acid solution.
Raise the temperature of the solution to 55 to 60° C.
Add TG (Transglucosidase enzyme (1.4 ml/kg wt. of dry starch) (Actually, 35 mL of solution is dosed with 166 μL of TG).
Incubate the solution in shaking water bath set at 55 to 60° C. for 24 hrs.
Take out samples from 16 hrs, 20 hrs and 24 hrs for DE measurements and oligosaccharide profile.
Terminate the reaction by pacing the solution in boiling water bath for 30 min.

### Scheme-B (3 Stages)

In some embodiments, the protocol of Scheme-B can comprise the following stages:

### Stage-1:

To 50 ml of BE solution, adjust the pH to 6.5 to 8.0 by using 3% Na₂CO₃.
Dose the solution with GlycoTransferase (Gtase—30 U/ml of starch) (Actually, 50 ml of solution requires 150 μL of GTase enzyme.
Incubate the reaction at 45 to 55° C. in shaking water bath for 60 min
Take out samples for DE and HPLC profile and proceed to Stage-2.

### Stage-2:

To solution from Stage-1, adjust pH to 4.5 to 5.5 by using a 3% Citric Acid solution.
Add Fungamyl enzyme (0.8 mL/Kg dry weight of starch) (Actually, 40 mL of Stage-1 solution is dosed with 12 μL of Fungamyl Enzyme).
Put the reaction solution in shaking water bath set at 50 to 55° C. Maintain the reaction at 50 to 55° C. for 20 min.
At exactly 20 min, terminate the reaction by placing the solution in boiling water bath for 30 min.
Cool the solution to room temperature and take out sample for HPLC-RI and DE analysis.
Proceed to Stage-3.

### Stage-3:

To Solution from Stage-2; adjust the pH to 5.0 to 5.5 using 3% citric acid solution.
Add TG (Transglucosidase enzyme (1.4 ml/kg wt. of dry starch) (Actually, 35 mL of Stage-2 solution is dosed with 166 μL of TG).
Incubate the solution in shaking water bath set at 55 to 60° C. for 24 hrs.
Take out samples from 16 hrs, 20 hrs and 24 hrs for DE measurements and oligosaccharide profile.
Terminate the reaction by pacing the solution in boiling water bath for 30 min.

### Scheme-C (4 Stages)

In some embodiments, the protocol of Scheme-C can comprise the following stages:

### Stage-1:

To 50 ml of BE solution, adjust the pH to 6.5 to 8.0 by using 3% Na₂CO₃.
Dose the solution with GlycoTransferase (Gtase—30 U/mL of starch) (Actually, 50 ml of post-BE solution requires 150 μL of GTase enzyme.
Incubate the reaction at 45 to 55° C. in shaking water bath for 60 min
Take out samples for DE and HPLC profile and proceed to Stage-2.

### Stage-2:

To solution from Stage-1, adjust pH to 4.5 to 5.5 by using a 3% Citric Acid solution.
Add Fungamyl enzyme (0.8 mL/Kg dry weight of starch) (Actually, 40 mL of Stage-1 solution is dosed with 12 μL of Fungamyl enzyme).
Put the reaction solution in shaking water bath set at 53° C. Maintain the reaction at 55 to 60° C. for 20 min.
At exactly 20 min, terminate the reaction by placing the solution in boiling water bath for 30 min.
Cool the solution to room temperature and take out sample for HPLC-RI and DE analysis.
Proceed to Stage-3.

### Stage-3:

To 50 ml of BE solution, adjust the pH to 5.5 to 6.0 by using 3% Na₂CO₃.
Dose the solution with Cycloglucanotransferase (CGTase—3.4 U/g of starch), (Actually, 35 mL of solution requires 3.5 g of CGTase enzyme).
Incubate the reaction at 88 to 90° C. in shaking water bath for 60 min.
Take out samples for DE and HPLC profile and proceed to Stage-4.

### Stage-4:

To Solution from Stage-3; adjust the pH to 5.0 to 5.5 by using a 3% citric acid solution.
Add TG (Transglucosidase enzyme (1.4 ml/kg wt. of dry starch) (Actually, 35 mL of solution is dosed with 166 μL of TG).
Incubate the solution in shaking water bath set at 55 to 60° C. for 24 hrs.
Take out samples from 16 hrs, 20 hrs and 24 hrs for DE measurements and oligosaccharide profile.
Terminate the reaction by pacing the solution in boiling water bath for 30 min.

### Scheme-D (3 Stages)

In some embodiments, the protocol of Scheme-D can comprise the following stages:

### Stage-1:

To 50 ml of post BE solution, adjust pH to 4.5 to 5.5 by using a 3% Citric Acid solution.
Add Fungamyl enzyme (0.8 mL/kg dry weight of starch) (Actually, 100 ml of BE step solution is dosed with 26 μL of Fungamyl Enzyme).
Put the reaction solution in shaking water bath set at 50 to 55° C. Maintain the reaction at 53° C. for 20 min.
At exactly 20 min, terminate the reaction by placing the solution in boiling water bath for 30 min.
Cool the solution to room temperature and take out sample for HPLC-RI and DE analysis.
Proceed to Stage-2.

### Stage-2:

To Solution from Stage-1, adjust the pH to 6.5 to 8.0 by using 3% Na₂CO₃.
Dose the solution with GlycoTransferase (Gtase—30 U/ml of starch) (Actually, 84 mL of solution requires 252 μL of GTase enzyme.
Incubate the reaction at 45 to 55° C. in shaking water bath for 60 min.
Take out samples for DE and HPLC profile and proceed to Stage-3.

### Stage-3:

To Solution from Stage-2; adjust the pH to 5.0 to 5.5 by using a 3% citric acid solution.
Add TG (Transglucosidase enzyme (1.4 ml/kg wt. of dry starch) (Actually, 73 mL of solution is dosed with 102 μL of TG).
Incubate the solution in shaking water bath set at 55 to 60° C. for 24 hrs.
Take out samples from 16 hrs, 20 hrs and 24 hrs for DE measurements and oligosaccharide profile.
Terminate the reaction by pacing the solution in boiling water bath for 30 min.

### Scheme-E (3 Stages)

In some embodiments, the protocol of Scheme-E can comprise the following stages:

### Stage-1:

To 50 ml of Post BE solution, adjust the pH to 6.5 to 8.0 by using 3% Na₂CO₃.
Dose the solution with GlycoTransferase (Gtase—30 U/ml of starch) (Actually, 100 mL of solution requires 300 μL of GTase enzyme.
Incubate the reaction at 45 to 55° C. in shaking water bath for 60 min
Take out samples for DE and HPLC profile and proceed to Stage-2.

### Stage-2

To solution from Stage-1, adjust the pH to 5.5 to 6.0 using 3% Na₂CO₃
Dose the solution with Cycloglucanotransferase (CGtase—3.4 U/gr of starch), (Actually, 76 mL of solution requires 2.23 g of CGTase enzyme).
Incubate the reaction at 80 to 90° C. in shaking water bath for 60 min.
Take out samples for DE and HPLC profile and proceed to Stage-3.

### Stage-3

To Solution from Stage-2; adjust the pH to 5.0 to 5.5 using 3% citric acid solution.
Add TG (Transglucosidase enzyme (1.4 ml/kg wt. of dry starch) (Actually, 64 mL of solution is dosed with 90 μL of TG).
Incubate the solution in shaking water bath set at 55 to 60° C. for 24 hrs.
Take out samples from 16 hrs, 20 hrs and 24 hrs for DE measurements and oligosaccharide profile.
Terminate the reaction by pacing the solution in boiling water bath for 30 min.

### Scheme-F (4 Stages)

In some embodiments, the protocol of Scheme-F can comprise the following stages:

### Stage-1

To 50 ml of Post BE solution, adjust the pH to 6.5 to 8.0 by using 3% Na₂CO₃.
Dose the solution with GlycoTransferase (GTase—30 U/ml of starch) (Actually, 100 mL of solution requires 300 μL of GTase enzyme.
Incubate the reaction at 45 to 55° C. in shaking water bath for 60 min.
Take out samples for DE and HPLC profile and proceed to Stage-2.

### Stage-2

To solution from Stage-1, adjust the pH to 5.5 to 6.0 by using 3% Na₂CO₃.
Dose the solution with Cycloglucanotransferase (CGTase—3.4 U/gr of starch), (Actually, 84 mL of solution requires 2.46 of CGTase enzyme).
Incubate the reaction at 80 to 90° C. in shaking water bath for 60 min.
Take out samples for DE and HPLC profile and proceed to Stage-3.

### Stage-3

To solution from stage-2, adjust pH to 4.5 to 5.5 by 3% Citric Acid solution.
Add Fungamyl enzyme (0.8 mL/Kg dry weight of starch) (Actually, 72 mL of stage-2 solution is dosed with 18.7 μL of Fungamyl Enzyme).
Put the reaction solution in shaking water bath set at 53° C. Maintain the reaction at 50 to 55° C. for 20 min.
At exactly 20 min, terminate the reaction by placing the solution in boiling water bath for 30 min.
Cool the solution to room temperature and take out sample for HPLC-RI and DE analysis.
Proceed to Stage-4.

### Stage-4

To Solution from Stage-3; adjust the pH to 5.0 to 5.5 using 3% citric acid solution.
Add TG (Transglucosidase enzyme (1.4 ml/kg wt. of dry starch) (Actually, 60 mL of solution is dosed with 84.3 μL of TG).
Incubate the solution in shaking water bath set at 55 to 60° C. for 24 hrs.
Take out samples from 16 hrs, 20 hrs and 24 hrs for DE measurements and oligosaccharide profile.
Terminate the reaction by pacing the solution in boiling water bath for 30 min.

### Scheme-G (4 Stages)

In some embodiments, the protocol of Scheme-F can comprise the following stages:

### Stage-1

To 50 ml of post BE solution, adjust pH to 4.5 to 5.5 by 3% Citric Acid solution.
Add Fungamyl enzyme (0.8 mL/Kg dry weight of starch) (Actually, 50 mL of BE step solution is dosed with 13 μL of Fungamyl Enzyme).
Put the reaction solution in shaking water bath set at 53° C. Maintain the reaction at 50 to 55° C. for 20 min.
At exactly 20 min, terminate the reaction by placing the solution in boiling water bath for 30 min.
Cool the solution to room temperature and take out sample for HPLC-RI and DE analysis.
Proceed to Stage-2.

### Stage-2

To solution from Stage-1, adjust the pH to 6.5 to 8.0 by using 3% Na₂CO₃.
Dose the solution with GlycoTransferase (Gtase—30 U/ml of starch) (Actually, 42.5 mL of solution requires 127 μL of GTase enzyme.
Incubate the reaction at 45 to 55° C. in shaking water bath for 60 min.
Take out samples for DE and HPLC profile and proceed to Stage-3.

### Stage-3

To solution from Stage-3, adjust the pH to 5.5 to 6.0 by using 3% Na2CO3
Dose the solution with Cycloglucanotransferase (CGTase—3.4 U/gr of starch), (Actually, 37.5 mL of solution requires 1.1 g of CGTase enzyme).
Incubate the reaction at 80 to 90° C. in shaking water bath for 60 min.
Take out samples for DE and HPLC profile and proceed to Stage-4.

### Stage-4

To Solution from Stage-3; adjust the pH to 5.0 to 5.5 using 3% citric acid solution.
Add TG (Transglucosidase enzyme (1.4 ml/kg wt. of dry starch) (Actually, 32 mL of solution is dosed with 15 μL of TG).
Incubate the solution in shaking water bath set at 55 to 60° C. for 24 hrs.
Take out samples from 16 hrs, 20 hrs and 24 hrs for DE measurements and oligosaccharide profile.
Terminate the reaction by pacing the solution in boiling water bath for 30 min.

During Fungamyl deactivation, Post Cycloglucanotransferase enzyme step and deactivation of Transglucosidase enzyme step, some gelatinous brownish precipitates may form in post reaction solutions. Those precipitate can be later removed by passing the solution through a celite filtration process and resultant was a clear, transparent brown colored solution.

### Comparison of Experimental Schemes

### Scheme-A:

In some embodiments, this scheme can comprise one of the simplest and shortest enzymatic schemes used herein and can be the foundation for all forthcoming enzymatic reactions. This scheme does not include the addition of any of new enzymes, i.e., GTase or CGTase. This scheme is intended to test the Fungamyle enzyme to Post-BE treated solution, before addition of TG enzyme. Since TG requires smaller chains for reactivity, mostly Maltose, Fungamyle can be used to facilitate that.

Scheme-A can be represented as: BE-Funga-TG

### Scheme-B:

In some embodiments, a new enzyme, GTase, can be added to Scheme-A right after BE step, to compare the action of GTase on post-BE solution, and also on final product.

Scheme-B can be represented as: BE-GTase-Funga-TG

and where Scheme-A can be represented as: BE-Funga-TG Scheme-C:

In some embodiments, another enzyme, CGTase, can be added to see its effect upon final product. The CGTase can be first added in this scheme after Fungamyle step, but later on in Scheme-F, can be added after GTase step. So, CGTase was tested at two different locations within the enzymetic schemes in order to find out its most suitable position.

Scheme-C can be represented as: BE-GTase-Funga-CGTase-TG

and where Scheme-F can be represented as: BE-GTase-CGTase-Funga-TG

### Scheme-D:

In some embodiments, this scheme can comprise a rearrangement of the enzymes as used in Scheme-B, i.e., GTase and Fungamyle were switched over. The purpose was to see if GTase can be more effective right after using of BE enzyme, and before using the Fungamyle, since Fungamyle breakdown the long chains into short chains, whereas GTase require at least 7-glucose chains to make a branching point within olio structure.

Scheme-D can be represented as: BE-Funga-GTase-TG

and where Scheme-B can be represented as: BE-GTase-Funga-TG

### Scheme-E:

In some embodiments, this scheme can comprise a modified form of Scheme-C, from which the Fungamyle enzyme was removed. This scheme was intended to see the effect of GTase and CGTase enzyme together upon BE-treated solution, and without addition of any Fungamyle at all. Later, this scheme was proved to be the best one in term of total Fiber value of the final product, as well as overall increase in oligo fraction >dp2.

Scheme-E can be represented as: BE-GTase-CGTase-TG

and where Scheme-C can be represented as: BE-GTase-Funga-CGTase-TG

### Scheme-F:

In some embodiments, this scheme is similar to that of Scheme-C, except for a re-arrangements of enzymes, and includes an additional enzyme step over Scheme-E, to see the addition of Fungamyle along with the together effect of GTase and CGTase. The purpose was to see if there is further increase in the total dietary fiber content in Post-TG sample out of this scheme.

Scheme-F can be represented as: BE-GTase-CGTase-Funga-TG

and where Scheme-C can be represented as: BE-GTase-Funga-CGTase-TG

### Scheme-G:

In some embodiments, this scheme can comprise a re-arrangement of enzymes used in Scheme-F, by moving the Fungamyle right after BE-step, and can be a modification of Scheme-D by the addition of CGTase. The purpose was to see the effect of Fungamyle location in those schemes and also effect of CGTase on overall Fiber value in Scheme-D.

Scheme-G can be represented as: BE-Funga-GTase-CGTase-TG

and where Scheme-F can be represented as: BE-GTase-CGTase-Funga-TG

and where Scheme-D can be represented: BE-Funga-GTase-TG

### Results and Data Analysis

### HPLC and Chromatograms

The Carbohydrate Profiles specific to IMO and IMO-R are obtained by running the samples on HPLC-RI system using Shodex column. Only the final samples after Transglucosidase (TG) reaction step were selected to confirm the identity of typical IMO, and run with a set of know standards, i.e. Glucose, Maltose, Isomaltose, Panose and Isomaltotriose, maltotetraose (dp4) including α-, β- & γ-cyclodextrins. A-cyclodextrin peak was found to be overlapped with that of dp4 standard peak, however, β-cyclodextrin, and γ-cyclodextrin peaks were distinctive.

The Oligosaccharide Profile were analyzed on HPLC-RI with Rezex column, and samples were run for all the post-enzymatic steps including; Branchzyme, GTase, CGTase, Fungamyle and TG steps. The representative Oligosaccharide Profiles, and Carbohydrate profiles from main enzymatic steps within each given schemes along with the brief explanation are given as follows:

Scheme-A: Full Chromatogram profile on HPLC with Phenomenex Rezex Column—Liquified starch treated with Branchzyme (BE) for up to 24 hrs.

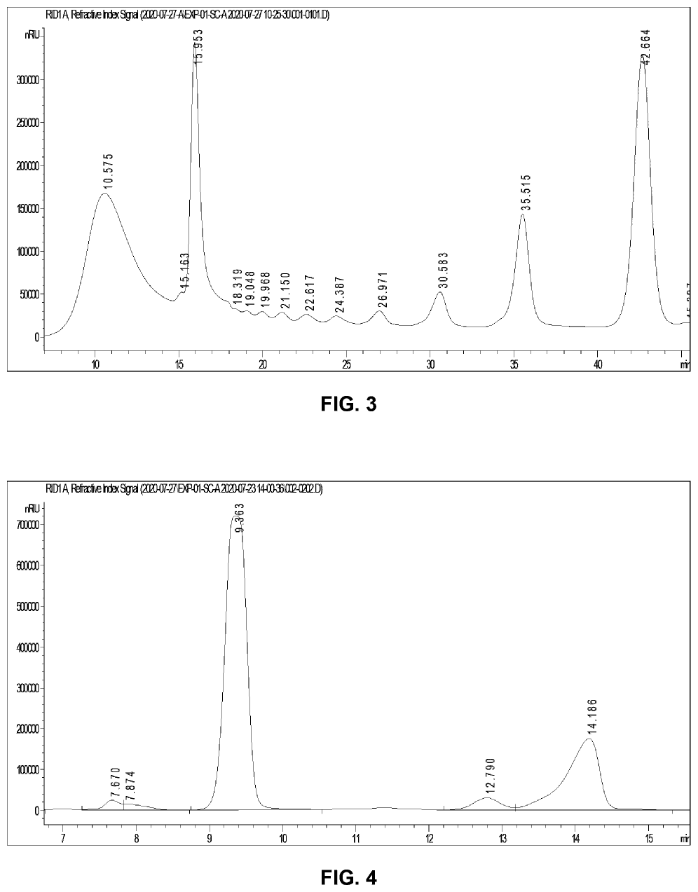

Scheme-A was conducted under two experimental conditions with different setup. FIG. 2 showing first part of the data, and with FIG. 3 showing the second part of data.

Liquified starch solution was treated with Branchzyme (BE) enzyme for 24 hrs, the resultant mixture showed minor breakdown (up to 10%) of starch glucose chains into oligosaccharide chains ranging from dp1 to dp9, and about 90% showed as a single large peak at RT=15.990. That observation suggests that BE in the absence of other hydrolytic enzymes, is unable to convert majority of the large glucose chains present in starch into shorter oligo chains. That observation is further supported by the following data.

[Scheme-A]: Full Chromatogram Profile on HPLC with Phenomenex Rezex Column—Liquified Starch First Treated with Branchzyme (BE), and then Treated with Transglucosidase Enzyme (TG).

Referring to FIG. 3, TG generated its characteristic high level of glucose in post-treated samples, which showed a big peak at RT=42.664. Even without addition of any hydrolytic enzyme, e.g., Fungamyle, a prominent increase can be seen in disaccharide content (RT=35.515) and little increase in other content. The large peak at RT=15.953 and 10.573 represent the presence of undigested larger molecular weight polysaccharide in the mixture. It is interesting to note that, the larger peak at RT-10.575 completely vanished in the presence of Fungamyle in our experiment, which apparently converted into smaller chains oligosaccharide with the increase in content of dp2 to dp8.

[Scheme-A]: Full Chromatogram Profile on HPLC with Shodex Column—Liquified Starch First Treated with Branchzyme (BE), and Finally Treated with Transglucosidase Enzyme (TG).

FIG. 4 displays the same sample as of FIG. 3, but on the Shodex column.

[Scheme-A]: Full Chromatogram Profile on HPLC with Phenomenex Rezex Column—Liquified Starch First Treated with Branchzyme (BE), and then Treated with Fungamyle Enzyme and Finally Treated with TG Enzyme. Following Profile is after the Fungamyle Step.

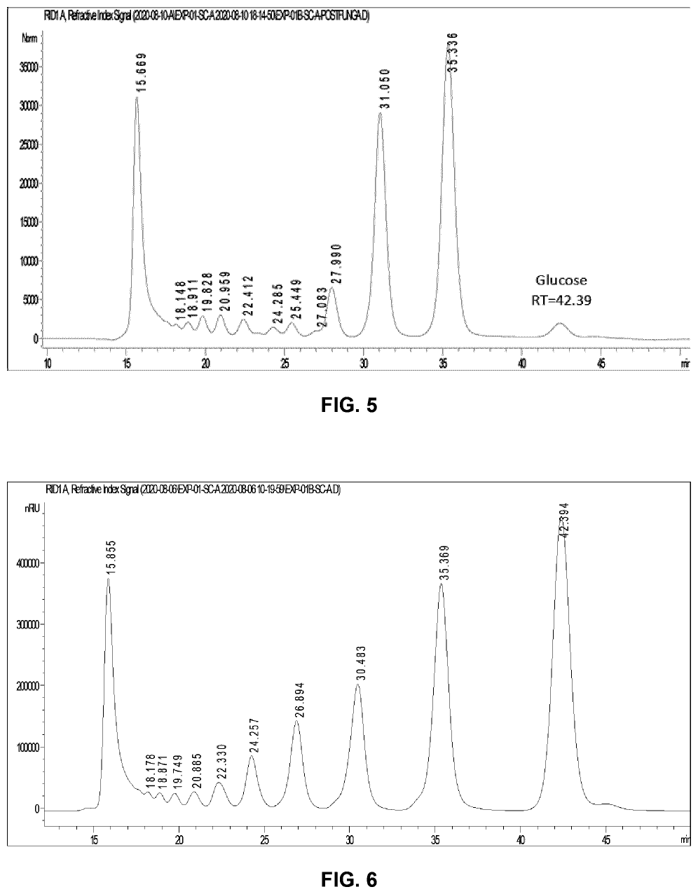

Purpose of this step was to evaluate the effect of Fungamyle enzyme in post-BE treated sample. As shown in FIG. 5, the Fungamyle enzyme caused a considerable increase in glucose (RT=42.39), dp2 and dp3 contents, which were not the case when this enzyme was absent (refer to FIGS. 2 and 3). Also, the large molecular weight peaks at RT=15.855 also greatly reduced, suggesting the facilitative role of Fungamyle in breaking down the larger polysaccharides into smaller oligosaccharide molecules. However, there wasn't observed any increased in the oligosaccharide portion.

[Scheme-A]: Full Chromatogram Profile on HPLC with Phenomenex Rezex Column—Liquified Starch First Treated with Branchzyme (BE), and then Treated with Fungamyle Enzyme and Finally with Transglucosidase (TG) Enzyme. Following Profile is after TG Step.

Referring to FIG. 6, the combination of Fungamyle and TG resulted into perfect generation of a typical smaller chain oligosaccharide product which is specific to IMO kind of product and is containing glucose peak at RT=42.394 to all the way to d10 at RT=18.178. The rest of oligo content, i.e., >dp10 are showed as a single peak at TR=15.855. These data showed a systematic and novel approach for generating isomalto-oligosacchride (IMO) mixture with the combined effects of BE enzyme, Fungamyle enzyme, and TG enzymes.

[Scheme-A]: Full Chromatogram Profile on HPLC with Shodex Column—Liquified Starch First Treated with Branchzyme (BE), and then Treated with Fungamyle Enzyme and Finally with Transglucosidase (TG) Enzyme. This Chromatogram is after TG Step and Taken on Shodex Column.

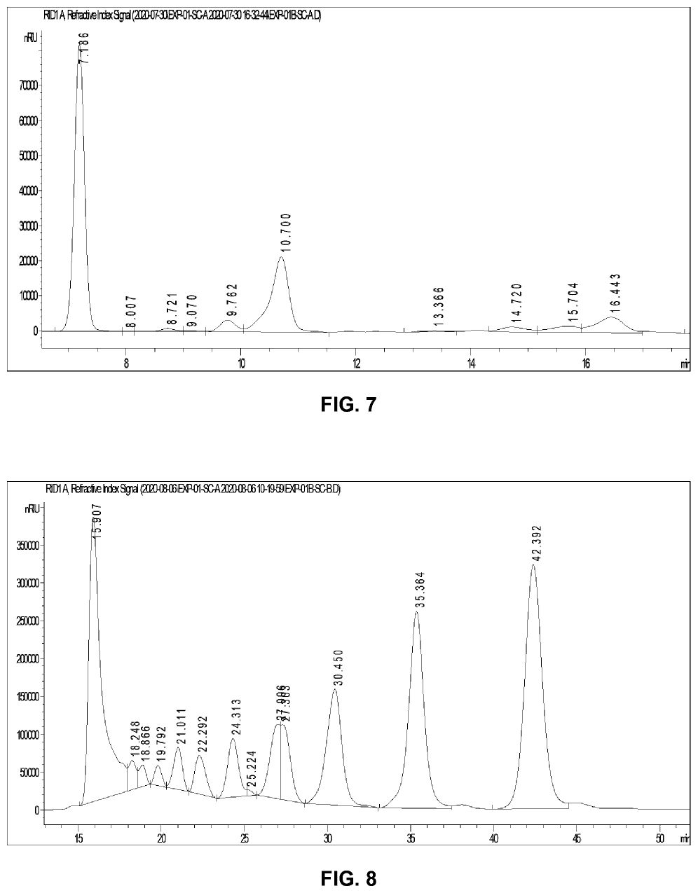

Referring to FIG. 7, this is the same sample as using in FIG. 6, but using a different, oligo Shodex column on HPLC. Column resolution was found to be lesser in our earlier samples, compared to the Rezex column, however, prominent peaks of glucose (RT=7.186), and dp2 (RT=10.7) can be seen, along with the dp3 peaks. In later attempt a more high-resolution data could be obtained which is given below with the samples from other schemes.

[Scheme-B]: Full Chromatogram Profile on HPLC with Phenomenex Rezex Column—Liquified Starch First Treated with Branchzyme (BE), and then Treated with GlycoTransferase (GTase), then Treated with Fungamyle Enzyme and Finally with Transglucosidase (TG) Enzyme. Following Profile is after Final TG Step.

Referring to FIG. 8, in this scheme, a new enzyme, i.e., GTase, was introduced first time, which resulted a new peak at RT=27.303 (adjacent to the dp4 fraction), and also resulted into increased in dp7 content (RT=21.011). This scheme was conducted in comparison with that of Scheme A, to see the effect of use of new enzyme in branching effects of IMO. These observations shows the extra branching takes place in the IMO mixture, because of the action of GTase enzyme, which is an α(1,3) bonding formation enzyme.

[Scheme-B]: Full Chromatogram Profile on HPLC with Showa Asahi Pack—Shodex Column—Liquified Starch First Treated with Branchzyme (BE), and then Treated with GlycoTransferase (GTase), then Treated with Fungamyle Enzyme and Finally with Transglucosidase (TG) Enzyme. Following Profile is after GTase Step.

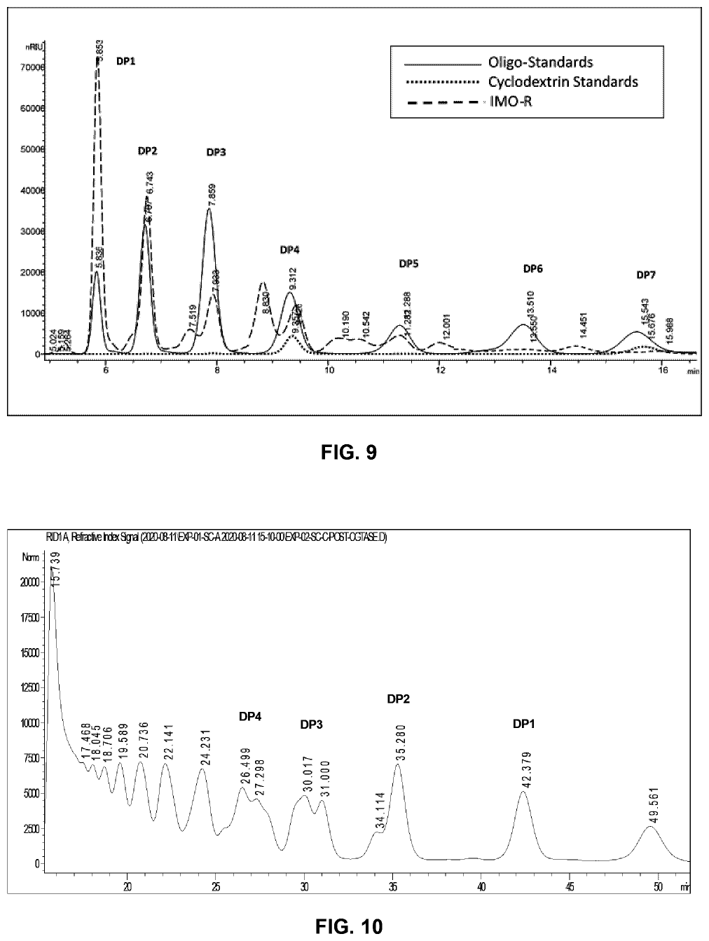

Referring to FIG. 9, this is the same sample as of FIG. 8, except it run on a different Shodex Oligo column. Profile showed here is overlapped with the two sets of standard: Oligo standards (Solid-line), and cyclo-dextrin standards (Dotted-line). Signature peaks for a typical IMO product (Dashed-line) are evident from DP2 (isomaltose), DP3 (Isomaltotriose) and DP4 (isomaltotetraose). This profile confirms the identity of product as ‘Isomalto-oligosaccharide’ or IMO.

[Scheme-C]: Full Chromatogram Profile on HPLC with Phenomenex Rezex Column—Liquified Starch First Treated with Branchzyme (BE), and then Treated with GlycoTransferase (GTase), then Treated with Fungamyle Enzyme, then Treated Cyclo-Glucanotransferase (CGTase), and Finally with Transglucosidase (TG) Enzyme. Following Spectra is after CGTase Enzyme Step.

Referring to FIG. 10, in this scheme, another new enzyme, CGTase, is first introduced in addition to GTase enzyme as per previous step. Enzyme CGTase addition further caused additional peaks at DP3, DP4 and DP5, which shows extensive branching within oligo structure.

[Scheme-C]: Full Chromatogram Profile on HPLC with Phenomenex Rezex Column—Liquified Starch First Treated with Branchzyme (BE), and then Treated with GlycoTransferase (GTase), then Treated with Fungamyle Enzyme, then Treated Cyclo-Glucanotransferase (CGTase), and Finally with Transglucosidase (TG) Enzyme.] Following Spectra is after the Final TG Step.

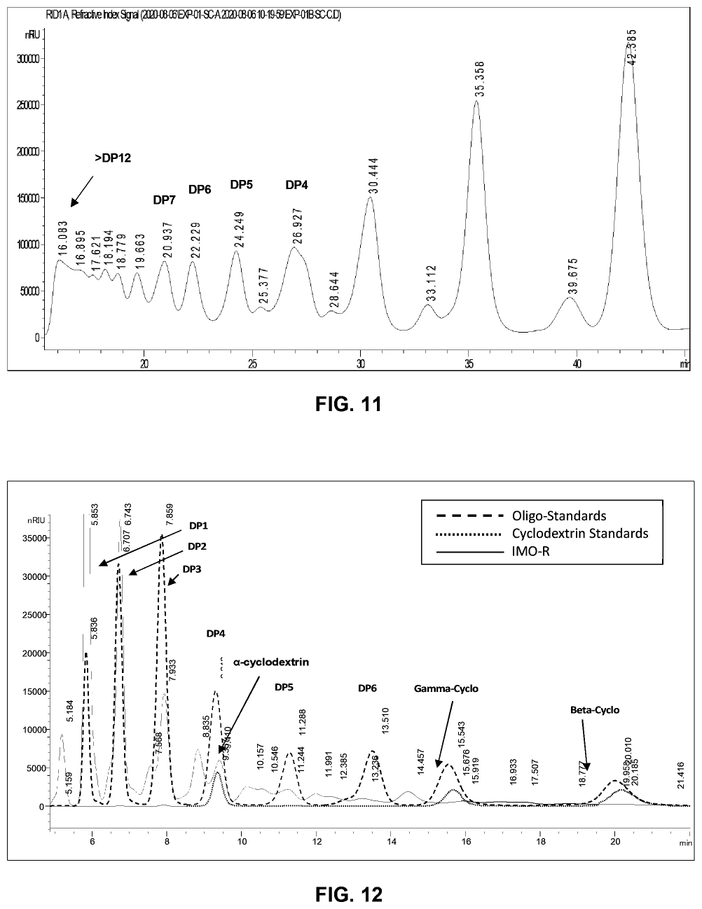

Referring to FIG. 11, shown is the combined effect of two additional new enzymes, i.e., GTase and CGTase along with BE in earlier step and TG in final step resulted an extensive branching within the oligo-structure. Note a peak of large oligos is mostly converted into smaller oligos with the increase in our all contents of dp4, dp5, dp6 and dp7, out of effect of BE, GTase, CGTase and TG enzymes.

[Scheme-C]: Full Chromatogram Profile on HPLC with Showa Asahi Pack—Shodex Column—Liquified Starch First Treated with Branchzyme (BE), and then Treated with GlycoTransferase (GTase), then Treated with Fungamyle Enzyme, then Treated Cyclo-Glucanotransferase (CGTase), and Finally with Transglucosidase (TG) Enzyme.] Following Spectra is after the Final TG Step.

Referring to FIG. 12, this is the same sample as FIG. 11, except run on Shodex oligo column. The sample was run with oligo-standards (Dash-line) and cyclodextrin standards (Dotted-line). IMO identification peaks (solid line) are evident in dp2, dp3, and dp4. Cyclic oligos formations are also showed at RT=9.312 for α-cyclodextrin; RT=15.676 for γ-cyclodextrin; and RT=20.185 for β-cyclodextrin.

[Scheme-D]: Full Chromatogram Profile on HPLC with Phenomenex Rezex Column—Liquified Starch First Treated with Branchzyme (BE), and then Treated with Fungamyle Enzyme, then Treated with GlycoTransferase (GTase), and Finally with Transglucosidase (TG) Enzyme. Following Spectra is after Final TG Step.

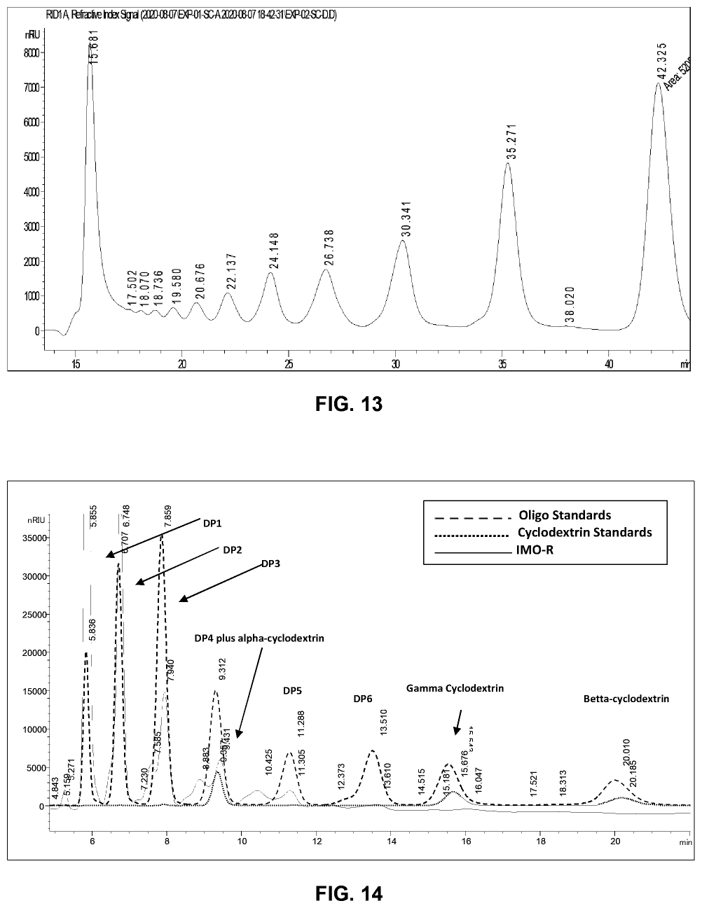

Referring to FIG. 13, this profile is in comparison with that of Scheme-B profile; the only difference is Fungamyle enzyme as used before the GTase enzyme in Scheme-D. The double peaks at DP4 area as showed in Scheme-B is not converted into one sharp peak here at RT=26.738. Also, the overall content of larger oligos in Scheme-B looks higher than Scheme-D. This suggests that GTase enzyme works well when added soon after the BE enzyme and before the Fungamyle enzyme.

[Scheme-D]: Full Chromatogram Profile on HPLC with Showa Asahi Pack—Shodex Column—Liquified Starch First Treated with Branchzyme (BE), and then Treated with Fungamyle Enzyme, then Treated with GlycoTransferase (GTase), and Finally with Transglucosidase (TG) Enzyme]. Following Profile is after Final TG Step.

Referring to FIG. 14, this is the same sample as of FIG. 13 but on Shodex column. Sample run with a set of oligo standards (Dash-line), and cyclo-dextrin standards (Dotted-line). Characteristic IMO peaks (solid line) evident at DP2 (isomaltose), DP3 (panose and iso-dp3), and DP4 (iso-dp4). Alpha and gamma cyclic dextrin generated from CGTase enzyme also showed at DP4 and DP7 at this level. This scheme established the basis of much branched IMO-R product.

[Scheme-E]: Full Chromatogram Profile on HPLC with Phenomenex Rezex Column—Liquified Starch First Treated with Branchzyme (BE), and then Treated with GlycoTransferase (GTase), then Treated with CGTase Enzyme, and Finally with Transglucosidase (TG) Enzyme]. Following Spectra is after Final TG Step.

Referring to FIG. 15, this profile is taken in comparison with that of Scheme-C, in which Funga enzyme was used between GTase and CGTase enzyme. In this Scheme-E, Funga enzyme didn't used. The results from Scheme-D showed the use of Funga somewhat reduced the higher oligo content, therefore effect of both new enzymes, i.e., GTase and CGTase were analyzed here. Despite no Funga was used, glucose content was prominent (RT=42.311) and higher oligos from Dp3, on words were significantly higher than any scheme so far. There is prominent increase in the content of Dp5 (RT=24.144), and doubling peak at Dp4 (RT=26.752) is completely converted into single component. The increase in Dp5 may be in result of formation of alpha-cyclodextrin with different combination of linkages.

[Scheme-E]: Full Chromatogram Profile on HPLC with Showa Asahi Pack—Shodax Column—Liquified Starch First Treated with Branchzyme (BE), and then Treated with with GlycoTransferase (GTase), then Treated with CGTase Enzyme, and Finally with Transglucosidase (TG) Enzyme]. Following Spectra is after Final TG Step. Same Sample as of FIG. 7, but on Shodex Oligo Column.

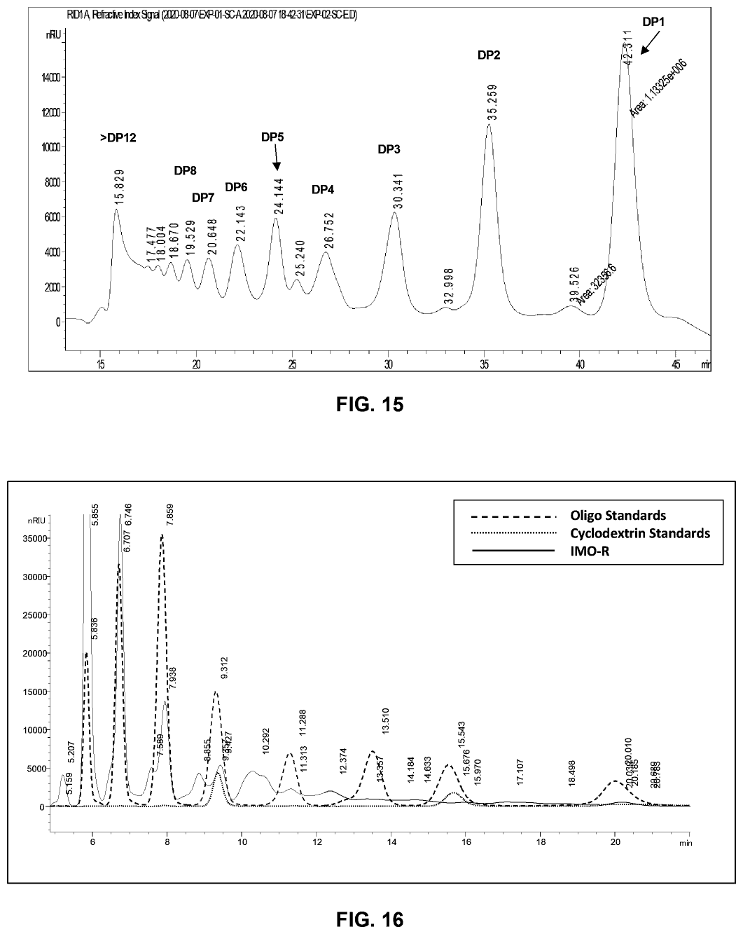

Referring to FIG. 16, IMO identification peaks (solid line) are evident for Dp2 at RT=6.747; for Dp3 at RT=7.859; for Dp4 at RT=9.312. The cyclodextrin product (dotted-line) also evident at RT=9.312, 15.543 and 20.185.

[Scheme-F]: Full Chromatogram Profile on HPLC with Phenomenex Rezex Column—Liquified Starch First Treated with Branchzyme (BE), and then Treated with with GlycoTransferase (GTase), then Treated with CGTase Enzyme, then Treated with Fungamyle, and Finally with Transglucosidase (TG) Enzyme. Following Spectra is after Final TG Step.

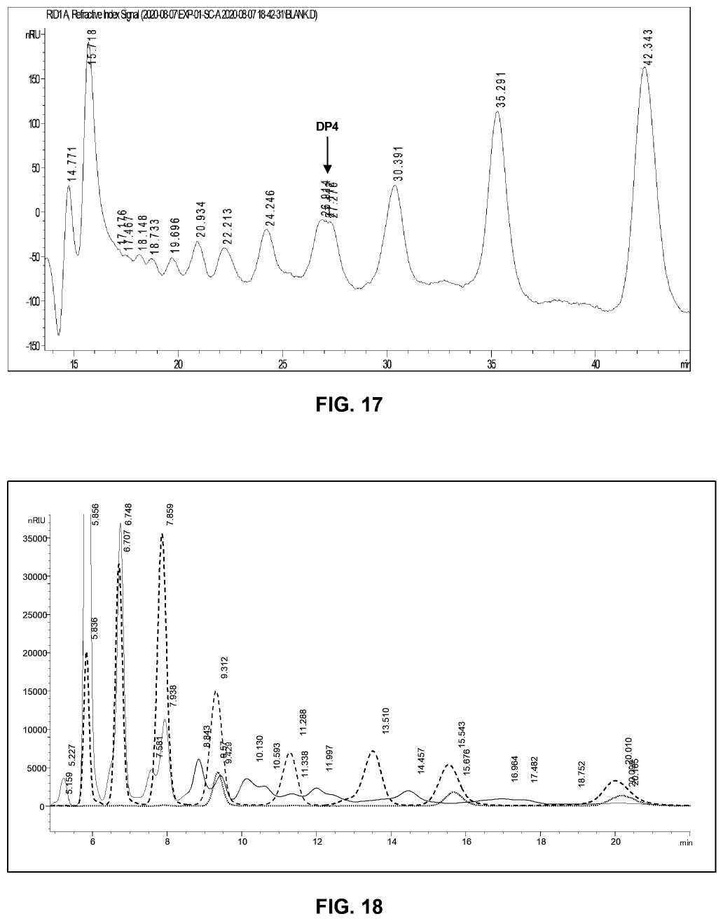

Referring to FIG. 17, the purpose of this scheme was to see the additional effect of Fungamyle enzyme using the same scheme of enzymes as of Scheme-E. It could be seeing that Dp4 fraction in Scheme-E was a single sharp peak, is now converted basically three equivalent peaks (RT=26.9-27.2), which suggest the extensive hydrolytic action of Fungamyle.

[Scheme-F]: Full Chromatogram Profile on HPLC with Shodex Column—Liquified Starch First Treated with Branchzyme (BE), and then Treated with GlycoTransferase (GTase), then Treated with CGTase Enzyme, then Treated with Fungamyle, and Finally with Transglucosidase (TG) Enzyme. Following Spectra is after Final TG Step, Using the Same Sample as that of FIG. 17, but Run on the Shodex Column.

Referring to FIG. 18, IMO identification peaks (solid line) are evident for Dp2 at RT=6.707; for Dp3 at RT=7.938; for Dp4 at RT=9.312. The peaks for larger oligos seems to shift either at left or right side of the respective standards peaks, probably because of the combination of different bonding within oligo structure. The cyclodextrin product (dotted-line) also evident at RT=9.312, 15.457 and 17.482.

[Scheme-G]: Full Chromatogram Profile on HPLC with Phenomenex Rezex Column—Liquified Starch First Treated with Branchzyme (BE), and then Treated with Fungamyle, then Treated with GlycoTransferase (GTase), then Treated with CGTase Enzyme, and Finally with Transglucosidase (TG) Enzyme].

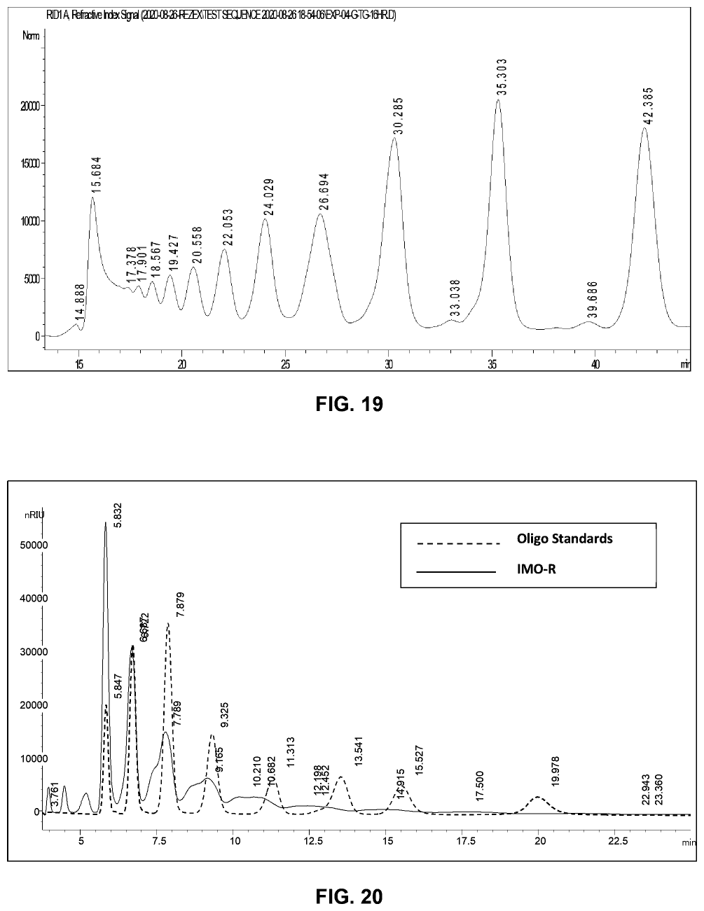

Referring to FIG. 19, the scheme was designed to test the effect of Fungamyle before the addition of GTase and CGTase in comparison of Scheme-F, in which the Fungamyle was added after these two enzymes. Glucose content (RT=42.385) was reduced and the three peaks at the level of Dp4, as observed in Scheme-F, was converted back into singe peak, as was in the case of Scheme-E. However, the increase in Dp5 was not equivalent to that of as obtained in Scheme-E. Overall, this scheme yields higher oligos content gradually decreased down as the dp's increased.

[Scheme-G]: Full Chromatogram Profile on HPLC Shodex Column—Liquified Starch First Treated with Branchzyme (BE), and then Treated with Fungamyle, then Treated with GlycoTransferase (GTase), then Treated with CGTase Enzyme, and Finally with Transglucosidase (TG) Enzyme. Same Sample as was Used in FIG. 19, but Run on Shodex Column for IMO Identification.

Referring to FIG. 20, shown is a full Chromatogram profile on HPLC with Showa Asahi pack—Shodex Column; IMO identification peaks are evident for Dp2 at RT=6.66; for Dp3 at RT=7.789; for Dp4 at RT=9.325. The peaks for larger oligos seems to shift either at left or right side of the respective standards peaks, probably because of the combination of different bonding within oligo structure.

The results of the final purified IMO-R product analysis including chromatographic profile from Rezex column (oligo-profile) and Shodex column (IMO-profile) are given as follows:

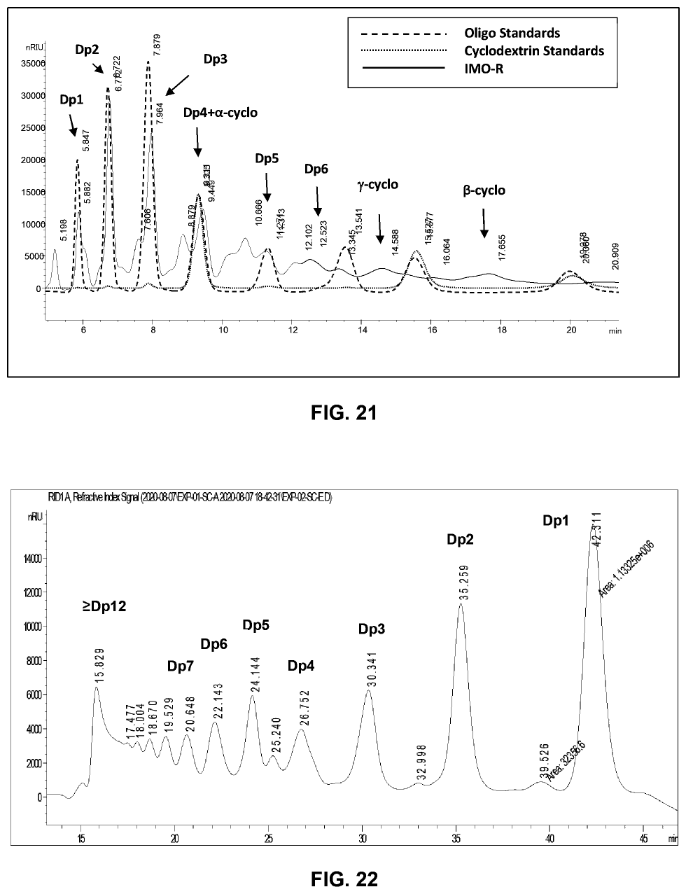

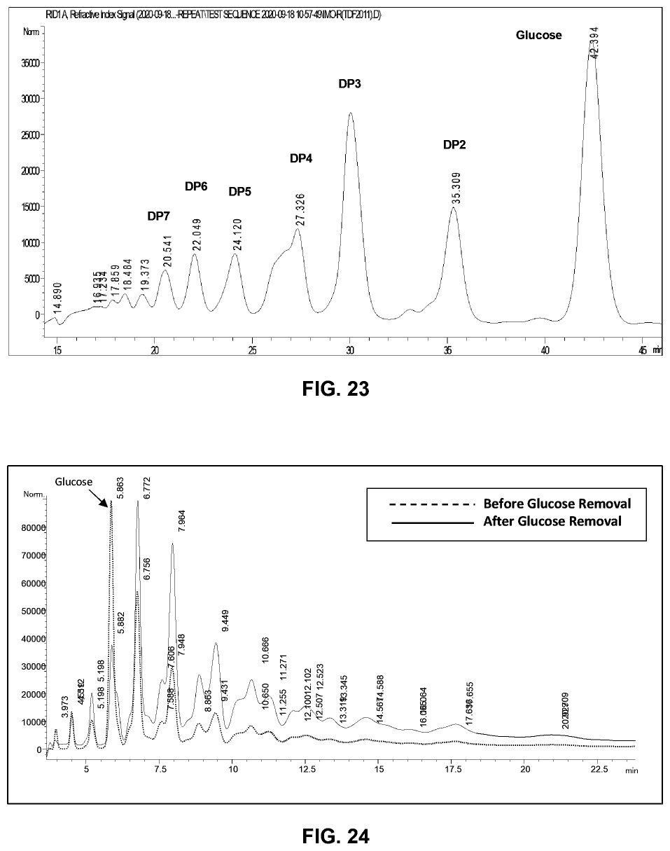

Referring to FIG. 21, shown is a full chromatogram profile on HPLC-RI with Showa Asahi pack—Shodex Column. Referring to FIG. 22, shown is a full chromatogram profile on HPLC with Phenomenex Zerbex Column. Referring to FIG. 23, shown is a full chromatogram profile on HPLC with Phenomenex Zerbex Column after treating the IMO-R with pancreatic alpha-amylase (PAA), and amyloglucosidase (AMG) for 16 hrs, as per protocol of AOAC-2011 standard method (in-house TDF testing). Referring to FIG. 24, shown is a comparison of Before and After Glucose filtration from Final IMO-R sample.

### Dextrose Equivalent (DE) Analysis

Dextrose equivalent (DE) value was determined by Lane Enyon Titration method. This method involves a titration of appropriately diluted liquefied starch or hydrolysate sample against a pre-standard Fehling Reagent under heat in the presence of Methylene Blue as an Indicator. Complete procedure with calculations is described in Appendix-3. Dextrose Equivalent results of various schemes are given in Table-2 shown below.

TABLE 2

| | | Process | | | | | |
|---|---|---|---|---|---|---|---|
| Enzymatic | | Temp | Process | Process | | DE | Expected |
| Schemes | Enzyme Used | (° C.) | pH | Time | | Value | Range |
| Scheme-A | Branchzyme | 63-67 | 6.3-6.7 | 24 | hr | 21.4 | 20-25 |
| | Fungamy1 800L | 50-55 | 4.5-5.5 | 20 | min | 37 | 30-35 |
| | Transglucosidase | 55-60 | 5.0-5.5 | 24 | hr | 56 | 50-55 |
| Scheme-B | Glyco Transferase (GTase) | 45-55 | 6.5-8.0 | 60 | min | 38 | 30-35 |
| | Fungamyl | 50-55 | 4.5-5.5 | 20 | min | 43 | 30-35 |
| | Transglucosidase (TG) | 55-60 | 5.0-5.5 | 24 | hr | 56 | 50-55 |
| Scheme-C | Glyco Transferase (GTase) | 45-55 | 6.5-8.0 | 60 | min | 39 | 30-35 |
| | Cycloglucono-transferase | 80-90 | 5.5-6.0 | 60 | min | 42 | 30-35 |
| | (CGTase) | | | | | | |
| | Fungamyl | 50-55 | 4.5-5.5 | 20 | min | 43 | 30-35 |
| | Transglucosidase (TG) | 55-60 | 5.0-5.5 | 24 | hr | 54 | 50-55 |
| Scheme-D | Fungamyl | 50-55 | 4.5-5.5 | 20 | min | 42 | 30-35 |
| | GTase | 45-55 | 6.5-8.0 | 60 | min | 44 | 30-35 |
| | Transglucosidase (TG) | 55-60 | 5.0-5.5 | 24 | hr | 57 | 50-55 |
| Scheme-E | Glyco Transferase (GTase) | 45-55 | 6.5-8.0 | 60 | min | 41 | 30-35 |
| | Cyclo-gluconotransferase | 80-90 | 5.5-6.0 | 60 | min | 43 | 30-35 |
| | (CGTase)- Toruzyme | | | | | | |
| | Transglucosidase (TG) | 55-60 | 5.0-5.5 | 16 | hr | 56 | 50-55 |
| Scheme-F | Cyclo-gluconotransferase | 80-90 | 5.5-6.0 | 60 | min | 41 | 30-35 |
| | (CGTase) | | | | | | |
| | Glyco Transferase (GTase) | 45-55 | 6.5-8.0 | 60 | min | 43 | 30-35 |
| | Fungamyl | 50-55 | 4.5-5.5 | 20 | min | 44 | 30-35 |
| | Transglucosidase (TG) | 55-60 | 5.0-5.5 | 16 | hr | 54 | 50-55 |
| Scheme-G | Fungamyl | 50-55 | 4.5-5.5 | 20 | min | 46 | 30-35 |
| | Glyco Transferase (GTase) | 45-55 | 6.5-8.0 | 60 | min | 47 | 30-35 |
| | Cycloglucono-transferase | 80-90 | 5.5-6.0 | 60 | min | 48 | 30-35 |
| | (CGTase) | | | | | | |
| | Transglucosidase (TG) | 55-60 | 5.0-5.5 | 16 | hr | 56 | 50-55 |

Italicized data are the final post-TG sample analysis

### Total Dietary Fiber (TDF) Analysis

Total Dietary Fiber is (TDF) measured by enzymatic reactions of Pancreatic α-amylase (PAA) and Amyloglucosidase (AMG), and analysis with HPLC-RI system. TDF is measured by standard AOAC-2011 and AOAC-2017 methods for the final post-TG sample generated from different enzymatic schemes employed in the current project. The HPLC-RI system was equipped with a Rezex oligo column for the analysis of post-digestive samples during the process. As the post enzymatic samples doesn't contain any Ash or Protein more than 1% combined, sample was, therefore, directly applied on HPLC. The peaks selected for the peak-areas count were from dp3 onwards, as anything before dp3 are not recognized as a fiber by the current regulatory rules.

For estimated calculations of TDF, following formula was used using the HPLC chromatogram and peak areas of the digested samples:

$$TDF ( approx . ) = 100 - ( Difference in Glucose content before and after amylase treatment + Maltose Content )$$

Note: The TDF data reported here is the approximation only, and based upon the respective peak areas percentage of post TDF procedures as per AOAC-2017 and AOAC-2011 standard methods. It is expecting that the data presented here should be near to the actual Fiber value. The in-house measured TDF data from 7-schemes are summarized in Table-3 set out below.

TABLE 3
Total Dietary Fiber Measurement (in-house) using Standard Methods

| Experiment # | Scheme | AOAC-2011 | AOAC-2017 | |
|---|---|---|---|---|
| Exp-01 | A | | 40.49 | BE-Funga-TG |
| | B | | 42.74 | BE-Gtase-Funga-TG |
| | C | | 45.18 | BE-Gtase-Funga-CGTase-TG |
| Exp-02 | D | 63.39 | 54.63 | BE-Funga-Gtase-TG |
| | E | 72.89 | 64.67 | BE-Gtase-CGTase-TG |
| | F | 70.55 | 57.97 | BE-Gtase-CGTase-Funga |
| Exp-03 | E (repeat) (#1) | | 70.16 | BE-Gtase(30U)-CGTase(0.3U)-TG |
| | E (Modi-1) | | 72.8 | BE-Gtase(40U)-CGTase(0.6U)-TG |
| | E (Modi-2) | | 72.6 | BE-Gtase(50U)-CGTase(0.9U)-TG |
| | F (repeat) | | 61.5 | BE-Gtase(30U)-CGTase(0.3U)-Funga |
| | F (Modi-1) | | 60.44 | BE-Gtase(40U)-CGTase(0.6U)-Funga |
| | F (Modi-2) | | 63.33 | BE-Gtase(50U)-CGTase(0.9)-Funga |
| Exp-04 | G | | 48.82 | BE-Funga-Gtase-CGTase |
| | E (repeat) (#2) | | 72.9 | BE-Gtase-Cgtase-TG |
| Exp-05 | E (repeat) (#3) | 62.0 | 51 | BE-Gtase-CGTase-TG |

Based upon above data, and chromatographic profiles given, out of 7-schemes the Scheme-E showed highest value of estimated TDF in the range of 51.0% to 73.0% when tested using AOAC-20011 method, and 62.0-72.89% when tested using the AOAC-2017 method. However, getting the averaged value of six TDF data obtained from AOAC-2011 method, and two data obtained from the AOAC-2017 method yielded the value of TDF as of 67.0%. To confirm our in-house measured TDF date, a sample was also submitted to a 3rd party lab (Merieux NutriSciences-Ontario) with the request using AOAC-2011 method. The TDF results came back from the 3rd party lab is given in Table 4 below, and Certificate of the is of Analysis same attached in Appendix-2

TABLE 4
Measurement of Total Dietary Fiber (TDF) by 3rd Party Lab.

| Sample | 3^{rd} Party Laboratory | Method Used | TDF Results |
|---|---|---|---|
| Scheme-E | Merieux NutriScience | AOAC-2011.25 | 51% |
| (Post Glucose Removal) | | | |

The reported value from the external lab (51%) showed 16% less than the average value (67%) obtained from our in-house TDF measurement. However, the external lab later confirmed that the value they reported was somewhat under-estimated as in their opinion because of non-suitability of the method used, namely there are some limitation specifically towards the measurement of correct TDF value for an IMO sample.

In post-sample testing communications with the external lab, the testing lab suggest further testing may needed using different standards method, i.e., AOAC-2001, and for which the Applicant is intended to do so. Regardless, the reported value of TDF, i.e., 51% is still the highest value of Fiber in any given IMO product in the market.

Above given data proved the IMO-R made in the current project by using novel enzymatic schemes produced an IMO product with much better digestion-resistant characteristics with highest possible dietary fiber value reported so far.

### Removal of Glucose

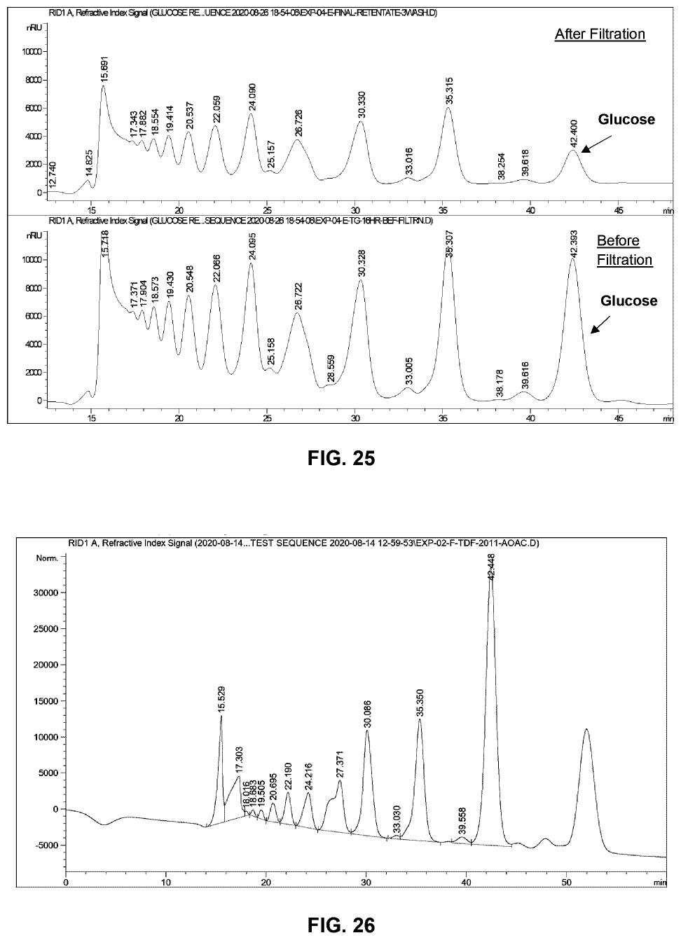

A high level of glucose (˜35 to 40%) is generated during enzymatic reactions, particularly after Fungamyle and Transglucosidase treatments in the current schemes. Presence of glucose considered as a contaminant in IMO/IMO-R product since that not only undermine the health claims of the product but also influenced upon overall dietary fiber measurement. Therefore, it become imperative to remove the excess glucose level out of final IMO-R product. For that purpose, a nano-filtration technology was employed in the current project, which basically involved passing the final product solution through a specific molecular-weight-cut-off (MWCO) membrane under pressure setup. A laboratory scale instrument was acquired from Sterlitech-USA (Model HP4750) equipped with a nano filtration membrane with MWCO of 600-800 Da. The device was run under N₂ pressure, and sample was washed with at least 4-5 times Sample-volume using pure water. The final post-filtration sample was concentrated using rotary evaporator to the solid content of 47% (from initial 18%). The overall glucose content in the sample before filtration start was 42.3% reduced, that reduced to 3.4% after 4 washings with pure water. The chromatographic profile from HPLC-Rezex column is shown in FIG. 25. A detailed experimental procedure is given in Appendix 5.

### CONCLUSIONS

Based upon the in-house Fiber analysis data, out of 7-schemes employed in the current studies, the Scheme-E showed the highest estimated Fiber value in the range of 51.0% to 73.0% when tested using standard AOAC-20011 method, and 62.0-72.89% when tested using the standard AOAC-2017 method. The averaged value out of six TDF data obtained from AOAC-2011 method, and two data obtained from the AOAC-2017 method yielded the value of TDF as of 67.0%.

The same sample from Scheme-E was also tested by a 3rd party external lab, which reported Fiber value as 51% using AOAC 2011 method. Later, the external Lab confirmed the reported value may not correct representation of all fiber content in IMO-R product because of the method limitations towards IMO type of products. Thus, we conclude that the Fiber value for IMO-R is in the range of 60% would be most suitable after scientifically comparing the in-house and external lab's averaged data.

Based upon in-house testing data, Scheme-F showed next to the best after Scheme E with the TDF content approximate in the range of about 50-60% as per in-house testing.

The DE measurement value showed the Dextrose Equivalent (DE) value of the final sample of almost all enzymatic protocols are found within the expected range, i.e. 50-55%.

The carbohydrate profile of Scheme-E product on HPLC-RI with Rezex column confirmed the high level of oligo contents greater than dp2. Also, increased branching and new peaks appearance are evident in fractions >dp3. That suggest the action of novel set of enzymes, i.e., BE, GTase and CGTase work well and as expected.

The carbohydrate profile of Scheme-E final product on HPLC-RI with Shodex column (and also final products from other schemes as well) confirmed the identification of isomalto-oligosaccharide (IMO), by having signature IMO's peaks of Isomaltose, Panose and Isomaltotriose components generated out of TG-enzyme action.

Large amount of glucose resulted from the post-TG enzyme action was successfully removed using a nano-filtration technique, in order to improve the TDF measurement

Although a few embodiments have been shown and described, it will be appreciated by those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

### REFERENCES

The following list of reference documents are incorporated by reference into this application in their entirety.
[1] Lee B-H, Yoo Y-H, Ryu J-H, et al. (2008) Heterologous expression and characterization of glycogen branching enzyme from Synechocystis sp. PCC6803. J Microbiol Biotechnol 18: 1386-1392.
[2] Lee B-H, Yan L, Phillips R J, et al. (2013) Enzyme-Synthesized Highly Branched Maltodextrins Have Slow Glucose Generation at the Mucosal α-Glucosidase Level and Are Slowly Digestible In Vivo. PLOS ONE 8(4): e59745. https://doi.org/10.1371/journal.pone.0059745.
[3] Mangas-Sanchez J and Adlercreutz P (2015) Enzymatic preparation of oligosaccharides by transglycosylation: A comparative study of glucosidases Author links open overlay panel. J Mol. Catal. B: Enzymatic 122:51-55.
[4] T. Hansson, P. Adlercreutz (2001) Enhanced transglucosylation/hydrolysis ratio of mutants of Pyrococcus furiosus β-glucosidase: Effects of donor concentration, water content, and temperature on activity and selectivity in hexanol. Biotech. Bioeng., 75 (2001), pp. 656-665.

### APPENDICES

1. Appendix 1: Dietary Fiber Analysis (TDF)
2. Appendix 2: Dietary Fiber—Certificate of Analysis by 3^{rd} Party Lab
3. Appendix 3: Dextrose Equivalent (DE) Determination
4. Appendix 4: HPLC Analysis Procedures
5. Appendix 5: Glucose Nanofiltration Procedure
6. Appendix 6: Starch Testing Method

### APPENDIX 1: Total Dietary Fiber (TDF) Analysis

Dietary Fiber Analysis is performed by two methods AOAC 2011 & 2017 which are available from Megazyme as kits for 100 samples analysis.

### AOAC 2017.16:

### Preparation of Solutions:

1. Sodium Maleate Buffer (50 mM, pH 6.0 plus 2 mM CaCl2 and 0.02% Sodium Azide): Dissolve 5.8 g of Maleic Acid in 800 mL of distilled water and adjust pH to 6.0 with 4M NaOH (16 g/100 mL) under stirring. Add 0.3 g of Calcium Chloride and 0.2 g of Sodium Azide and mix thoroughly to yield a clear solution.
2. Preparation of Enzyme PAA/AMG (Pancreatic α-amylase/Amyloglucosidase) solution for Digestion: Dissolve 0.2 g of PAA/AMG powder into a 15 mL Centrifuge Tube and add 10 ml of Sodium Maleate buffer (prepares for 7 tests of Dietary Fiber). Vortex the solution to completely dissolve the enzyme. Solution might be a little foamy on top. This enzyme solution should be used within 4 h of preparation.
3. Tris Buffer Solution (0.75M): Add 9.08 g of Tris Buffer Salt in 80 mL of de-ionized water to completely dissolve by stirring. Adjust the pH to 11.0 using NaOH, Make up the solution to 100 mL in a 150 ml bottle.
4. Preparation of Test Samples:

Use 0.25 g of sample for Dietary Fiber enzyme digestion and dilute according to the solid content measured by Refractometer. For example, if the sample solution has a solid content 30%, add 0.833 mL of solution to account for 0.25 g of product. Add 0.75 mL of sample solution into a 15 mL Centrifuge Tube and add 8.75 mL of Sodium Maleate buffer plus 1.25 mL of PAA/AMG mixture solution and cap the tubes and mix slowly by inverting the tube 2-3 times. Then the samples are incubated in a shaking water bath at 120 rpm at 370 C for exactly 4 hrs.
5. After the enzyme digestion, add 0.75 ml of Tris Buffer to adjust pH to 8.2 and place the tubes in boiling water bath >900 C for 30 min to deactivate the enzyme. Solution is cooled to room temperature.
6. 1.5 mL of this above step 5 solution is passed through Strong Cation and Anion Exchange resin cartridges (Orochem) and filtered through 0.2 μM syringe filters. Then these samples can be directly used for HPLC analysis.
7. Protein precipitation of the solution is attempted initially by taking small amount (1 mL) of sample and adding 18 mL of 95% ethanol. But no precipitation is observed as well even for non-digestible carbohydrates. Thus, the product contains mainly soluble oligosaccharides with less protein content. So Dietary Fiber results directly come from the HPLC analysis.
8. HPLC analysis is performed by Phenomenex Rezex Size exclusion column by Agilent 1100 HPLC equipped with Refractive Index Detector. HPLC conditions include the Column temperature at 80° C., RI detector at 35° C., Mobile phase-pure LC-MS water, injection volume 10 μL and run time 60 min.
9. From HPLC chromatogram, Dietary Fiber is the total of all peak area percentages starting from DP3 till the higher oligosaccharides.

### AOAC 2011:

### Preparation of Solutions:

1. Sodium Maleate Buffer (50 mM, pH 6.0 plus 2 mM CaCl2 and 0.02% Sodium Azide): Dissolve 5.8 g of Maleic Acid in 800 mL of distilled water and adjust pH to 6.0 with 4M NaOH (16 g/100 mL) under stirring. Add 0.3 g of Calcium Chloride and 0.2 g of Sodium Azide and mix thoroughly to yield a clear solution.
2. Preparation of Enzyme PAA/AMG (Pancreatic Alpha Amylase/Amido Glucosidase) solution for Digestion:

Dissolve 0.15 g of purified Porcine Pancreatic alpha-amylase into a 500 mL flask and add 290 ml of Sodium Maleate Buffer (50 mM) and stir for 5 min. Add 0.3 mL of AMG solution into the mixture and mix thoroughly to yield a clear solution. This solution should be stored at −100 C and thawed to room temperature whenever required for enzyme reactions.
3. Tris Buffer Solution (0.75M):

Add 9.08 g of Tris Buffer Salt in 80 mL of de-ionized water to completely dissolve by stirring. Make up the solution to 100 mL in a 150 ml bottle.
4. Preparation of Test Samples:

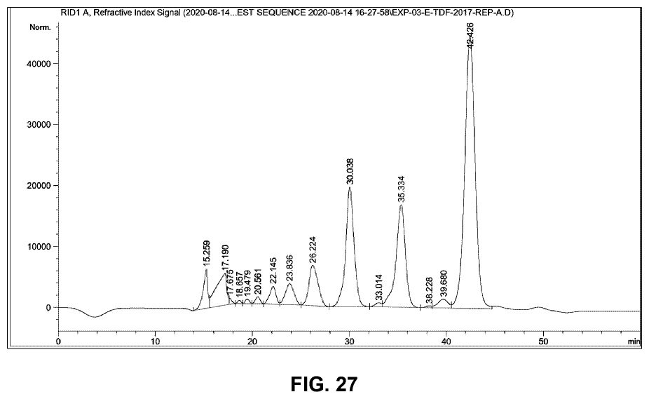

Use 0.25 g of sample for Dietary Fiber enzyme digestion and dilute according to the solid content measured by Refractometer. For example, if the sample solution has a solid content 30%, add 0.833 mL of solution to account for 0.25 g of product. Add 0.75 mL of sample solution into a 15 mL Centrifuge Tube and add 10 mL of PAA/AMG mixture solution and cap the tubes and mix slowly by inverting the tube 2-3 times. Then the samples are incubated in a shaking water bath at 120 rpm at 370 C for exactly 16 hrs.
5. After the enzyme digestion, add 0.75 mL of Tris Buffer to adjust pH to 8.2 and place the tubes in boiling water bath >900 C for 30 min to deactivate the enzyme. Solution is cooled to room temperature.
6. 1.5 mL of this above step 5 solution is passed through Strong Cation and Anion Exchange resin cartridges (Orochem) and filtered through 0.2 μM syringe filters. Then these samples can be directly used for HPLC analysis.
7. Protein precipitation of the solution is attempted initially by taking small amount (1 mL) of sample and adding 18 mL of 95% ethanol. But no precipitation is observed as well even for non-digestible carbohydrates. Thus, the product contains mainly soluble oligosaccharides with less protein content. So Dietary Fiber results directly come from the HPLC analysis.
8. HPLC analysis is performed by Phenomenex Rezex Size exclusion column by Agilent 1100 HPLC equipped with Refractive Index Detector. HPLC conditions include the Column temperature at 80° C., RI detector at 35° C., Mobile phase—pure LC-MS water, injection volume 10 μL and run time 60 min.
9. From HPLC chromatogram, Dietary Fiber is the total of all peak area percentages starting from DP3 till the higher oligosaccharides.
   HPLC-RI (with Rezex-Column) TDF profiles of sample after post-amylase hydrolysis using AOAC-2011 method is shown in FIG. 26.
   HPLC-RI (with Rezex-Column) TDF profiles of sample after post-amylase hydrolysis using AOAC-2017 method is shown in FIG. 27.

1. A method for preparing a resistant-isomalto-oligosaccharide (“IMO-R”) with increasing resistance to human gut enzymes, the method comprising:
a) adjusting the pH of a solution of liquified starch to about 4.5 to 5.5;
b) then adding an effective amount of Fungamyle 800L to the solution;
c) then incubating the solution at about 50 to 55 degrees Celsius for about 30 minutes, during which the solution is shaken or stirred;
d) then terminating activity of the Fungamyle 800L by raising the temperature of the solution to that of boiling water for about 30 minutes;
e) then cooling the solution to room temperature;
f) then adjusting the pH of the solution to about 5.0 to 5.5;
g) then adding an effective amount of Transglucosidase (“TG”) to the solution;
h) then incubating the solution at about 55 to 60 degrees Celsius for about 24 hours, during which the solution is shaken or stirred; and
i) then terminating activity of the TG by raising the temperature of the solution to that of boiling water for about 30 minutes.

2. The method as set forth in claim 1, wherein the effective amount of the Fungamyle 800L comprises 0.8 millilitre thereof per kilogram of dry weight of starch.

3. The method as set forth in claim 1, wherein the effective amount of the TG comprises 1.4 millilitre thereof per kilogram of dry weight of starch.

4. The method as set forth in claim 1, wherein the solution of liquified starch is prepared by:
a) mixing tapioca starch with water to form a starch slurry that is about 35% weight by volume;
b) then adjusting the pH of the starch slurry to about 6.3 to 6.7 using a Na₂CO₃ solution dosed with an effective amount of Kleistase;
c) then incubating the starch slurry at a temperature of about 87 degrees Celsius wherein an enzymatic reaction commences in the starch slurry;
d) then checking the starch slurry for its Dextrose Equivalent (“DE”);
e) then terminating the enzymatic reaction when the starch slurry is at a desired DE, wherein the enzymatic reaction is terminated by incubating the starch slurry at boiling water temperature for about 30 minutes and then cooling the starch slurry to room temperature;
f) then adding an effective amount of Branchzyme (“BE”) to the starch slurry;
g) then incubating the starch slurry by stirring it at about 63 to 67 degrees Celsius for about 24 hours;
h) then terminating activity of the BE when the starch slurry achieves a DE of 20 by raising the temperature of the starch slurry to that of boiling water for about 30 minutes; and
i) then cooling the starch slurry to room temperature thereby producing the solution of liquified starch.

5. The method as set forth in claim 4, wherein the effective amount of the Kleistase comprises 0.04% (v/w) thereof per gram of dry weight of starch.

6. The method as set forth in claim 4, wherein the effective amount of the BE comprises 600 Units thereof per gram of dry weight of starch.

7. The method as set forth in claim 1, prior to step a) thereof, further comprising:
a) adjusting the pH of the solution to about 6.5 to 8.0;
b) then adding an effective amount of GlycoTransferase (“GTase”) to the solution; and
c) then incubating the solution at about 45 to 55 degrees Celsius for about 60 minutes, during which the solution is shaken or stirred.

8. The method as set forth in claim 7, wherein the effective amount of the GTase comprises 30 Units thereof per millilitre of the solution.

9. The method as set forth in claim 1, prior to step a) thereof, further comprising:
a) adjusting the pH of the solution to about 6.5 to 8.0, then adding an effective amount of Glyco Transferase (“GTase”) to the solution; then incubating the solution at about 45 to 55 degrees Celsius for about 60 minutes, during which the solution is shaken or stirred; and
b) then, following step e) of claim 1, adjusting the pH of the solution to about 5.0 to 6.0, then adding an effective amount of Cycloglucanosyletransferase (“CGTase”) to the solution, and then incubating the solution at about 80 to 90 degrees Celsius for about 60 minutes, during which the solution is shaken or stirred.

10. The method as set forth in claim 9, wherein the effective amount of the GTase comprises 30 Units thereof per millilitre of the solution.

11. The method as set forth in claim 9, wherein the effective amount of the CGTase comprises 3.4 Units thereof per gram of dry weight of starch.

12. The method as set forth in claim 1, following step e) thereof, further comprising: adjusting the pH of the solution to about 6.5 to 8.0, then adding an effective amount of GlycoTransferase (“GTase”) to the solution, and then incubating the solution at about 45 to 55 degrees Celsius for about 60 minutes, during which the solution is shaken or stirred.

13. The method as set forth in claim 12, wherein the effective amount of the GTase comprises 30 Units thereof per millilitre of the solution.

14. The method as set forth in claim 12 following the steps thereof and prior to step f) of claim 1, further comprising:
a) adjusting the pH of the solution to about 5.0 to 6.0;
b) then adding an effective amount of Cycloglucanosyletransferase (“CGTase”) to the solution; and
c) then incubating the solution at about 80 to 90 degrees Celsius for about 60 minutes, during which the solution is shaken or stirred.

15. The method as set forth in claim 1, prior to step a) thereof, further comprising: adjusting the pH of the solution to about 6.5 to 8.0, then adding an effective amount of GlycoTransferase (“GTase”) to the solution, then incubating the solution at about 45 to 55 degrees Celsius for about 60 minutes, during which the solution is shaken or stirred, then adding an effective amount of Cycloglucanosyletransferase (“CGTase”) to the solution, and then incubating the solution at about 80 to 90 degrees Celsius for about 60 minutes, during which the solution is shaken or stirred.

16. The method as set forth in claim 15, wherein the effective amount of the GTase comprises 30 Units thereof per millilitre of the solution.

17. The method as set forth in claim 15, wherein the effective amount of the CGTase comprises 3.4 Units thereof per gram of dry weight of starch.

18. (canceled)

19. (canceled)

20. (canceled)

21. (canceled)

22. (canceled)

23. (canceled)

24. (canceled)

25. The method as set forth in claim 1, wherein the human gut enzymes comprise one or more of dextranase, α-glucosidase and α-amylase.

26. A human gut enzyme resistant isomalto-oligosaccharide (“IMO-R”), as produced by a method comprising:
a) adjusting the pH of a solution of liquified starch to about 4.5 to 5.5;
b) then adding an effective amount of Fungamyle 800L to the solution;
c) then incubating the solution at about 50 to 55 degrees Celsius for about 30 minutes, during which the solution is shaken or stirred;
d) then terminating activity of the Fungamyle 800L by raising the temperature of the solution to that of boiling water for about 30 minutes;
e) then cooling the solution to room temperature;
f) then adjusting the pH of the solution to about 5.0 to 5.5;
g) then adding an effective amount of Transglucosidase (“TG”) to the solution;
h) then incubating the solution at about 55 to 60 degrees Celsius for about 24 hours, during which the solution is shaken or stirred; and
i) then terminating activity of the TG by raising the temperature of the solution to that of boiling water for about 30 minutes.

27. The IMO-R as set forth in claim 26, wherein the effective amount of the Fungamyle 800L comprises 0.8 millilitre thereof per kilogram of dry weight of starch.

28. The IMO-R as set forth in claim 26, wherein the effective amount of the TG comprises 1.4 millilitre thereof per kilogram of dry weight of starch.

29. The IMO-R as set forth in claim 26, wherein the solution of liquified starch is prepared by:
a) mixing tapioca starch with water to form a starch slurry that is about 35% weight by volume;
b) then adjusting the pH of the starch slurry to about 6.3 to 6.7 using a Na₂CO₃ solution dosed with an effective amount of Kleistase;
c) then incubating the starch slurry at a temperature of about 87 degrees Celsius wherein an enzymatic reaction commences in the starch slurry;
d) then checking the starch slurry for its Dextrose Equivalent (“DE”);
e) then terminating the enzymatic reaction when the starch slurry is at a desired DE, wherein the enzymatic reaction is terminated by incubating the starch slurry at boiling water temperature for about 30 minutes and then cooling the starch slurry to room temperature;
f) then adding an effective amount of Branchzyme (“BE”) to the starch slurry;
g) then incubating the starch slurry by stirring it at about 63 to 67 degrees Celsius for about 24 hours;
h) then terminating activity of the BE when the starch slurry achieves a DE of 20 by raising the temperature of the starch slurry to about 90 degrees Celsius for about 30 minutes; and
i) then cooling the starch slurry to room temperature thereby producing the solution of liquified starch.

30. The IMO-R as set forth in claim 29, wherein the effective amount of the Kleistase comprises 0.04% (v/w) thereof per gram of dry weight of starch.

31. The IMO-R as set forth in claim 29, wherein the effective amount of the BE comprises 600 Units thereof per gram of dry weight of starch.

32. The IMO-R as set forth in claim 26, prior to step a) thereof, further comprising: adjusting the pH of the solution to about 6.5 to 8.0, then adding an effective amount of GlycoTransferase (“GTase”) to the solution, and then incubating the solution at about 45 to 55 degrees Celsius for about 60 minutes, during which the solution is shaken or stirred.

33. The IMO-R as set forth in claim 32, wherein the effective amount of the GTase comprises 30 Units thereof per millilitre of the solution.

34. The IMO-R as set forth in claim 26, prior to step a) thereof, further comprising:
a) adjusting the pH of the solution to about 6.5 to 8.0, then adding an effective amount of Glyco Transferase (“GTase”) to the solution, then incubating the solution at about 45 to 55 degrees Celsius for about 60 minutes, during which the solution is shaken or stirred; and
b) then, between steps e) and f) of claim 26, adjusting the pH of the solution to about 5.0 to 6.0, then adding an effective amount of Cycloglucanosyletransferase (“CGTase”) to the solution, and then incubating the solution at about 80 to 90 degrees Celsius for about 60 minutes, during which the solution is shaken or stirred.

35. The IMO-R as set forth in claim 34, wherein the effective amount of the GTase comprises 30 Units thereof per millilitre of the solution.

36. The IMO-R as set forth in claim 34, wherein the effective amount of the CGTase comprises 3.4 Units thereof per gram of dry weight of starch.

37. The IMO-R as set forth in claim 26, following step e) thereof, further comprising:
a) adjusting the pH of the solution to about 6.5 to 8.0;
b) then adding an effective amount of GlycoTransferase (“GTase”) to the solution; and
c) then incubating the solution at about 45 to 55 degrees Celsius for about 60 minutes, during which the solution is shaken or stirred.

38. The IMO-R as set forth in claim 37, wherein the effective amount of the GTase comprises 30 Units thereof per millilitre of the solution.

39. The IMO-R as set forth in claim 37 following the steps thereof and prior to step f) of claim 26, further comprising:
a) adjusting the pH of the solution to about 5.0 to 6.0;
b) then adding an effective amount of Cycloglucanosyletransferase (“CGTase”) to the solution; and
c) then incubating the solution at about 80 to 90 degrees Celsius for about 60 minutes, during which the solution is shaken or stirred.

40. The IMO-R as set forth in claim 26, prior to step a) thereof, further comprising: adjusting the pH of the solution to about 6.5 to 8.0, then adding an effective amount of GlycoTransferase (“GTase”) to the solution, then incubating the solution at about 45 to 55 degrees Celsius for about 60 minutes, during which the solution is shaken or stirred, then adding an effective amount of Cycloglucanosyletransferase (“CGTase”) to the solution, and then incubating the solution at about 80 to 90 degrees Celsius for about 60 minutes, during which the solution is shaken or stirred.

41. The IMO-R as set forth in claim 40, wherein the effective amount of the GTase comprises 30 Units thereof per millilitre of the solution.

42. The IMO-R as set forth in claim 40, wherein the effective amount of the CGTase comprises 3.4 Units thereof per gram of dry weight of starch.

43. (canceled)

44. (canceled)

45. (canceled)

46. (canceled)

47. (canceled)

48. (canceled)

49. (canceled)

50. (canceled)

51. (canceled)

52. (canceled)
